(19) United States
(12) Patent Application Publication
(10) Pub. No.: US 2025/0049799 A1
(43) Pub. Date: Feb. 13, 2025

(54) Targeted Therapy of Pyrrolo[2,3-D]Pyrimidine Antifolates in a Syngeneic Mouse Model of High Grade Serous Ovarian Cancer

(71) Applicants: Duquesne University of The Holy Spirit, Pittsburgh, PA (US); Wayne State University, Detroit, MI (US)

(72) Inventors: Aleem Gangjee, Allison Park, PA (US); Larry H. Matherly, Novi, MI (US)

(21) Appl. No.: 18/709,005

(22) PCT Filed: Nov. 9, 2022
(86) PCT No.: PCT/US2022/079542

§ 371 Date: May 10, 2024

Related U.S. Application Data
(60) Provisional application No. 63/263,922, filed on Nov. 11, 2021.

(51) Int. Cl. A61K 31/519; A61K 47/26; A61P 35/00

(52) U.S. Cl. CPC A61K 31/519; A61K 47/26; A61P 35/00

(57) ABSTRACT

A method of inhibiting M2-like macrophages in a patient is provided comprising administering to a patient having ovarian cancer a therapeutically effective amount of a FRB-transported C1 inhibitor compound having selective efficacy to FRβ expressing tumor cells. The method includes wherein the FRB-transported C1 inhibitor compound is a substituted pyrrolo[2,3-d]pyrimidine antifolate inhibitor compound having selective efficacy to FRβ expressing tumor cells. The present invention provides a method for targeting both the tumor and the tumor microenvironment with the FRB-transported C1 inhibitor compounds.

### CROSS-REFERENCE TO RELATED APPLICATIONS

This application is the United States national phase of International Application No. PCT/US2022/079542 filed Nov. 9, 2022, and claims the benefit of U.S. Provisional Patent Application No. 63/263,922, filed on Nov. 11, 2021, the disclosures of which are hereby incorporated by reference in their entireties.

### STATEMENT REGARDING FEDERALLY SPONSORED RESEARCH OR DEVELOPMENT

This invention was made with government support under Grant Nos. R01 CA53535, R01 CA166711, and R01 CA250469 awarded by the National Cancer Institute. The government has certain rights in the invention.

### BACKGROUND OF THE INVENTION

### 1. Field of the Invention

The present invention provides the therapeutic advantages of cytosolic C1 6-substituted pyrrolo[2,3-d]pyrimidine inhibitor compounds, for example but not limited to, the compounds having the structure of AGF94, AGF 278, and AGF283, with selectivity for uptake by FRs and PCFT and inhibition of de novo purine nucleotide biosynthesis, against a syngeneic model of ovarian cancer (BR-Luc) which recapitulates high-grade serous ovarian cancer in patients. In vitro activity of AGF94 was extended in vivo against orthotopic BR-Luc tumors. With late-stage subcutaneous Br-Luc xenografts, we demonstrate that AGF94 resulted in substantial anti-tumor efficacy accompanied by significantly decreased M2-like FRB-expressing macrophages and increased CD3+ T cells, whereas CD4+ and CD8+ T cells were unaffected. The methods of this invention provide potent anti-tumor efficacy of 6-substituted pyrrolo[2,3-d]pyrimidine inhibitor compounds in the therapy of epithelial ovarian cancer (EOC) in the context of an intact immune system and provide a framework for targeting the immunosuppressive tumor microenvironment (TME) as an essential component of patient therapy.

### 2. Background Art

Novel therapies are urgently needed for epithelial ovarian cancer (EOC), the most lethal gynecologic malignancy. In addition, therapies that target unique vulnerabilities in the tumor microenvironment (TME) of EOC have largely been unrealized. The present invention achieves selective drug delivery for EOC therapy using targeted antifolates via their uptake by folate receptor (FR) proteins, resulting in inhibition of essential one-carbon (C1) metabolic pathways. FRa is highly expressed in approximately 85% of EOCs, along with the proton-coupled folate transporter (PCFT); FRβ is expressed on activated macrophages, a major infiltrating immune population in EOC. Thus, there is great potential for targeting both the tumor and the TME with agents delivered via selective transport by FRs and PCFT.

### SUMMARY OF THE INVENTION

In certain embodiments of this invention, a method is provided of inhibiting M2-like macrophages in a patient comprising administering to a patient having ovarian cancer a therapeutically effective amount of a substituted pyrrolo[2,3-d]pyrimidine antifolate inhibitor having selective efficacy to FRβ expressing tumor cells. This method further includes in certain embodiments wherein the substituted pyrrolo[2,3-d]pyrimidine antifolate inhibitor is a compound selected from the group consisting of the following structures 1-12:

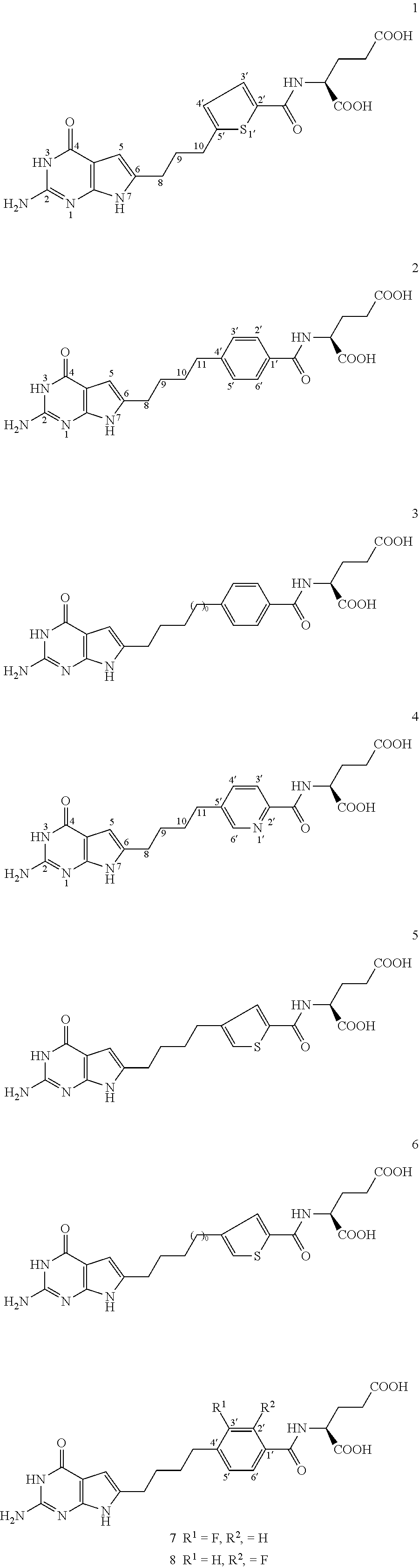

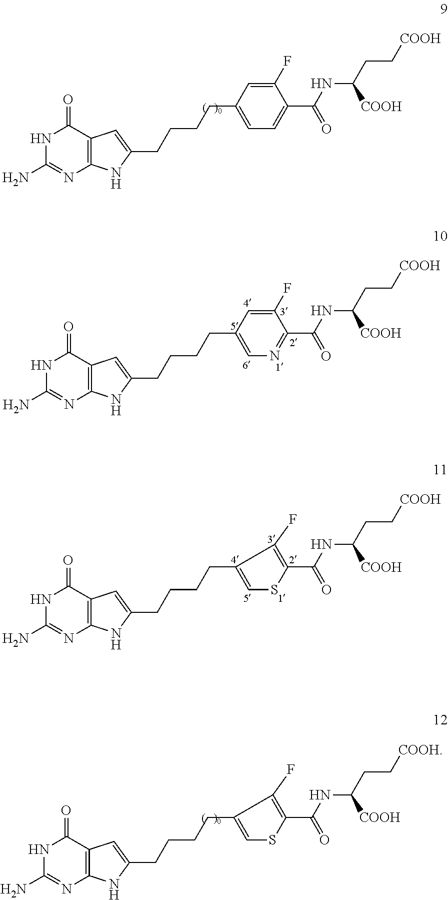

In certain preferred embodiments of this method, the compound has the structure 3, 11, or 12, above. In certain other embodiments of this method, the M2-like macrophage is an M2-like FRβ-expressing macrophage, including for example, wherein the M2-like FRβ-expressing macrophage is a tumor-associated macrophage (TAM).

In other embodiments of this invention, the method includes wherein the administration of the compound results in anti-tumor efficacy accompanied by decreased M2-like FRβ-expressing macrophages and increased CD3+ T cells, and wherein CD4+ and CD8+ T cells are unaffected.

In other embodiments, the method includes effecting anti-tumor efficacy of the compound in the therapy of epithelial ovarian cancer (EOC) in an intact immune system and targeting an immunosuppressive tumor microenvironment (TME).

In another embodiment of this invention, a method of inhibiting M2-like macrophages in a patient is provided comprising administering to a patient having ovarian cancer a therapeutically effective amount of a composition comprising a compound that is a substituted pyrrolo[2,3-d]pyrimidine antifolate inhibitor having selective efficacy to FRβ expressing tumor cells, and an acceptable pharmaceutical carrier. The method includes wherein the substituted pyrrolo[2,3-d]pyrimidine antifolate inhibitor is a compound selected from the group consisting of the compound structures 1-12 set forth herein. The method includes wherein the acceptable pharmaceutical carrier is one selected from the group of saline, dextrose and water, and sucrose. The method further includes wherein the composition includes one or more pharmaceutically acceptable excipients, fillers, binders, and surfactants. The method includes effecting anti-tumor efficacy of the composition in the therapy of epithelial ovarian cancer (EOC) in an intact immune system and targeting an immunosuppressive tumor microenvironment (TME).

In another embodiment of this invention, a method of inhibiting M2-like macrophages in a patient is provided comprising administering to a patient having ovarian cancer a therapeutically effective amount of a FRβ-transported C1 inhibitor compound having selective efficacy to FRB expressing tumor cells. This method includes wherein the FRβ-transported C1 inhibitor compound is a substituted pyrrolo[2,3-d]pyrimidine antifolate inhibitor compound having selective efficacy to FRβ expressing tumor cells. The method includes wherein the substituted pyrrolo[2,3-d]pyrimidine compound is a compound selected from the group consisting of the compound structures 1-12 set forth herein. This method includes effecting anti-tumor efficacy of said compound in the therapy of epithelial ovarian cancer (EOC) in an intact immune system and targeting an immunosuppressive tumor microenvironment (TME).

Another embodiment of this invention provides a method of inhibiting M2-like macrophages in a patient comprising administering to a patient having ovarian cancer a therapeutically effective amount of a composition comprising a FRβ-transported C1 inhibitor compound having selective efficacy to FRβ expressing tumor cells and a pharmaceutically acceptable carrier. The method includes wherein the pharmaceutically acceptable carrier is one selected from the group of saline, dextrose and water, and sucrose. This method further includes wherein the composition includes one or more pharmaceutically acceptable excipients, fillers, binders, and surfactants.

In certain other embodiments of this invention, a FRβ-transported C1 inhibitor compound having selective efficacy to FRβ expressing tumor cells for use in inhibiting M2-like macrophages for treating a patient having ovarian cancer, is provided. The FRβ-transported C1 inhibitor compound having selective efficacy to FRβ expressing tumor cells is selected from the group consisting of substituted pyrrolo[2,3-d]pyrimidine antifolate inhibitor compounds having selective efficacy to FRβ expressing tumor cells. The FRβ-transported C1 inhibitor compound having selective efficacy to FRβ expressing tumor cells that is a substituted pyrrolo[2,3-d]pyrimidine antifolate inhibitor compound is selected from the group consisting of the compound structures 1-12 set forth herein.

### BRIEF DESCRIPTION OF THE DRAWINGS

FIG. 1 shows structures of examples of 6-substituted pyrrolo[2,3-d]pyrmidine inhibitors of de novo purine nucleotide biosynthesis that are employed in the method(s) of this invention.

FIG. 2A shows expression of GARFTase transcripts in primary epithelial ovarian cancer (EOC) patient samples.

FIG. 2B shows expression of AICARFTase transcripts in primary EOC patient samples.

FIG. 2C shows transcript levels of cytosolic GARFTase and AICARFTase C1 metabolic targets in the BR-5 syngeneic mouse models of HGSOC by real-time RT-PCR.

FIG. 2D shows transcript levels of cytosolic GARFTase and AICARFTase C1 metabolic targets in the Br-Luc syngeneic mouse models of HGSOC by real-time RT-PCR.

FIG. 3A shows folate transporter expression in BR-Luc syngeneic HGSOC mouse models.

FIG. 3B shows folate transporter expression in BR-5 syngeneic HGSOC mouse models.

FIG. 3C shows total surface FRa that was measured by titration with [³H]folic acid at 0° C. with and without unlabeled 10 μmol/L non-radioactive folic acid.

FIG. 3D shows PCFT uptake that was assayed using [3H]MTX (0.5 μM) at pH 5.5 at 37° C. in the absence and presence of 10 μmol/L non-radiolabeled AGF94.

FIG. 4A shows AGF94 efficacy trial overall survival in IP BR-Luc model.

FIG. 4B shows a treatment scheme for the AGF94 efficacy trial of FIG. 4A.

FIG. 4C shows luminescent images for IP BR-Luc tumors in FVB mice (right panel of FIG. 4C) 1 day following treatment with AGF94 (32 mg/kg×4 doses) and control mice (left panel of FIG. 4C).

FIG. 5A shows luminescent images of AGF94 efficacy in a subcutaneous BR-Luc model.

FIG. 5B shows a trial design schematic for the AGF94 efficacy trial of FIG. 5A.

FIG. 5C shows plotted results for the BR-Luc trial efficacy arm with AGF94 by individual mice.

FIG. 5D shows a table that summarizes the results of the in vivo trial with SC BR-Luc xenografts treated with AGF94 for mice maintained on both the folate-deficient and folate replete diets.

FIG. 6A shows the impact of AGF94 treatment on tumor infiltrating macrophages, namely the percentage of CD11b+ and F4/80+ macrophages.

FIG. 6B shows the percentage of FRβ+, gated off CD11b+ and F4/80+ macrophages following 2, 3, or 4 doses of AGF94.

FIG. 6C shows the percentage of Arg1+FRβ+, gated off CD11b+ and F4/80+ macrophages, following 2, 3 or 4 doses of AGF94.

FIG. 6D shows the percentages of CD80+FRβ+, gated off CD11b+ and F4/80+ macrophages following 2, 3, or 4 doses of AGF94.

FIG. 7A shows the impact of AGF94 treatment on tumor infiltrating T cells, namely, percentages of CD3+ T cells from single cell suspensions of tumor and infiltrating lymphocytes following 2, 3 and 4 doses of AGF94.

FIG. 7B shows percentage of CD4+ T cells are shown from single cell suspensions of tumor and infiltrating lymphocytes following 2, 3 and 4 doses of AGF94.

FIG. 7C shows percentage of CD8+ T cells are shown from single cell suspensions of tumor and infiltrating lymphocytes following 2, 3 and 4 doses of AGF94.

FIG. 8 shows immunofluorescence staining of immune populations. FIG. 8 top panels are controls. FIG. 8 bottom panels are AGF94 treated mice tumors with the immunofluorescence staining for tumor sections stained with CD3 (FIG. 8 bottom left panel), CD8 (FIG. 8 bottom middle panel), or FRβ (red) antibodies (FIG. 8 bottom right panel) with DAPI counterstain (blue) (scale bars, 20 μm).

FIG. 9 shows the negative control for immunofluorescence staining shown in FIG. 8.

FIG. 10 shows a Schematic 1 having example structures of substituted pyrrolo[2,3-d]pyrimidine compounds of the method of this invention.

### DETAILED DESCRIPTION OF THE INVENTION

Epithelial ovarian cancer (EOC) remains the most lethal gynecologic malignancy, accounting for nearly 14,000 deaths yearly in the United States¹. The high mortality-to-incidence ratio for EOC is largely due to the development of resistance to standard cytotoxic chemotherapy in late-stage disease².

Increasing attention is focusing on targeted therapies for EOC mediated through folate receptors (FRs). FRa is expressed in ˜85% of EOCs, prompting development of FRa-targeted antibody drug conjugates (mirvetuximab soravtansine), cytotoxic folic acid drug conjugates [Vintafolide (EC145)], and targeted antifolates (ONX-0801)³⁻⁵. Several of these agents are in various stages of clinical development. With the discovery of the proton-coupled folate transporter (PCFT; SLC46A1) and its high level expression in solid tumors including EOC, attention has turned to the possibility of using PCFT for therapeutic targeting and several pyrrolo[2,3-d]pyrimidine antifolate inhibitors have been developed with selectivity for transport by both PCFT and FRa over the reduced folate carrier (RFC; SLC19A1), the major tissue folate transporter^{6, 7}.

The tumor microenvironment (TME) has emerged as a key determinant of disease progression and response to therapy in high grade serous ovarian cancer (HGSOC), the most common subtype of EOC⁸. The peritoneal spread of HGSOC creates a unique TME that promotes a complex interplay between the malignant ascites and surrounding tissues, allowing for tumor progression and immune evasion⁸. The predominant innate immune cellular component in ovarian cancer-associated ascites is the tumor-associated macrophage (TAM) population, which contributes to an immunosuppressive environment^{9, 10}. TAMs also play an important role in metastasis and angiogenesis by releasing proangiogenic factors (e.g., vascular endothelial growth factor, matrix metalloproteinase)^{11, 12}. Thus, inhibiting TAMs could in principle suppress tumor progression. Interestingly, ovarian cancer-associated TAMs express FRβ, affording an opportunity to inhibit TAMs via the selective uptake of FR-targeted therapeutics^{13, 14}.

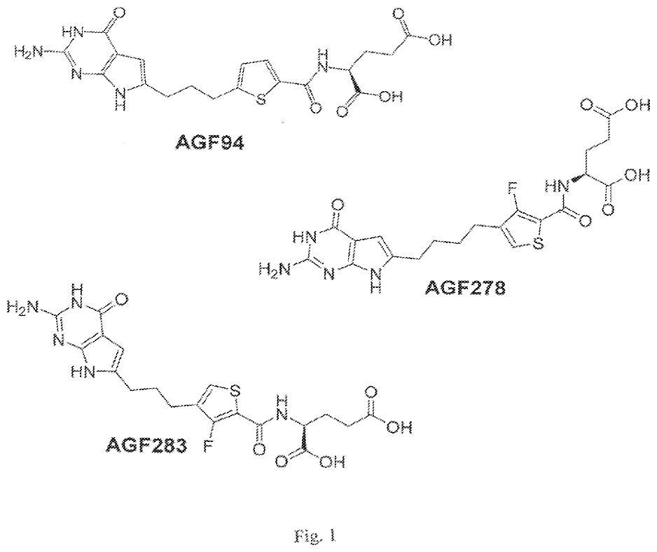

De novo purine biosynthesis is a critical pathway in tumor cells as purine depletion limits ATP and GTP for DNA synthesis and repair, and for cellular energetics. Our laboratory previously described novel 6-substituted pyrrolo[2,3-d]pyrimidine antifolate compounds that inhibit one-carbon (C1) metabolism in de novo purine biosynthesis, resulting in potent in vitro anti-tumor efficacy in tumor models including EOC^{6, 15, 16}. While substantial in vivo efficacy was reported in these studies with EOC xenografts in immune-compromised mice^{6, 15}, anti-tumor activity in the presence of an intact immune system was not explored. Our lead analogs (i.e., AGF94, AGF278, and AGF283; FIG. 1) inhibited glycinamide ribonucleotide (GAR) formyltransferase (GARFTase), the first folate-dependent step in the 10 reactions comprising de novo purine biosynthesis, resulting in perturbations in purine precursors [i.e., increased GAR, decreased formyl GAR and 5-aminoimidazole-4-carboxamide ribonucleotide (AICAR)] and decreased pools of purine nucleotides^{6, 15, 16}. Anti-tumor activity of this series of compounds including EOC reflects their endocytosis by FRa and/or their facilitative uptake by PCFT^{6, 15}.

To directly examine the impact of an intact immune system on the anti-tumor efficacy of our pyrrolo[2,3-d]pyrimidine inhibitors, we used a novel syngeneic mouse model of HGSOC (BR-Luc)^{17, 18}. The BR-Luc model was developed from the BR-5 EOC subline and both models are characterized by knockout of BRCA1 and p53, although BR-Luc cells also express a luciferase reporter^{17, 18}. Importantly, the BR-Luc EOC model recapitulates human HGSOC histology and patterns of metastasis, as well as responses to therapy¹⁸. We used the BR-Luc syngeneic model to investigate the relation between anti-tumor efficacy and the presence of immune infiltrates, including the impact on FRβ-expressing TAMs and CD3+, CD4+ and CD8+ T-cells, accompanying treatment with the pyrrolo[2,3-d]pyrimidine antifolate inhibitor AGF94. Our results are impactful in that they further establish the translational potential of this novel series of compounds against a clinically relevant syngeneic model of HGSOC. Interestingly, they also provide proof-of-concept that targeting M2-like macrophages in the TME of HGSOC via FRB contributes to in vivo efficacy of FR-targeted inhibitors of this class.

As used herein, the term “patient” means members of the animal kingdom, including but not limited to, human beings.

As used herein, the term “effective amount” or “therapeutically effective amount” refers to that amount of any of the present compounds, salts thereof, and/or compositions required to bring about a desired effect in a patient. The desired effect will vary depending upon the illness or disease state being treated. For example, the desired effect may be reducing the tumor size, destroying cancerous cells, and/or preventing metastasis, any one of which may be the desired therapeutic response. On its most basic level, a therapeutically effective amount is that amount of a substance needed to inhibit mitosis of a cancerous cell. As used herein, “tumor” refers to an abnormal growth of cells or tissues of the malignant type, unless otherwise specifically indicated and does not include a benign type tissue. The “tumor” may be comprised of at least one cell and/or tissue. The term “inhibits or inhibiting” as used herein means reducing growth/replication. As used herein, the term “cancer” refers to any type of cancer, including for example but not limited to, epithelial ovarian cancer, and the like.

The methods and novel compounds and pharmaceutically acceptable salts thereof of this invention provide for treatment of tumors, or other cancer cells, in cancer patients. The types of cancer can vary widely and in certain embodiments, the methods and novel compounds and pharmaceutically acceptable salts thereof of this invention are particularly useful for example, in treating epithelial ovarian cancer (EOC).

The compounds of the present invention are known compounds and the synthesis thereof is in the literature.

As used herein, the term “therapeutically effective carrier” refers to any pharmaceutically acceptable carrier known in the art, absent compatibility problems with the novel compounds of the invention. Generally, carriers include for example but not limited to, physiologic saline and 5% dextrose in water.

As will be understood by one skilled in the art, a therapeutically effective amount of said compound can be administered by any means known in the art, including but not limited to, injection, parenterally, intravenously, intraperitoneally, orally or, where appropriate, topically.

It is well within the skill of one practicing in the art to determine what dosage, and the frequency of this dosage, which will constitute a therapeutically effective amount for each individual patient, depending on the severity or progression of cancer or cancer cells and/or the type of cancer. It is also within the skill of one practicing in the art to select the most appropriate method of administering the compounds based upon the needs of each patient.

In certain embodiments of this invention, a method is provided of inhibiting M2-like macrophages in a patient comprising administering to a patient having ovarian cancer a therapeutically effective amount of a substituted pyrrolo[2,3-d]pyrimidine antifolate inhibitor having selective efficacy to FRβ expressing tumor cells. This method further includes in certain embodiments wherein the substituted pyrrolo[2,3-d]pyrimidine antifolate inhibitor is a compound selected from the group consisting of the following structures 1-12:

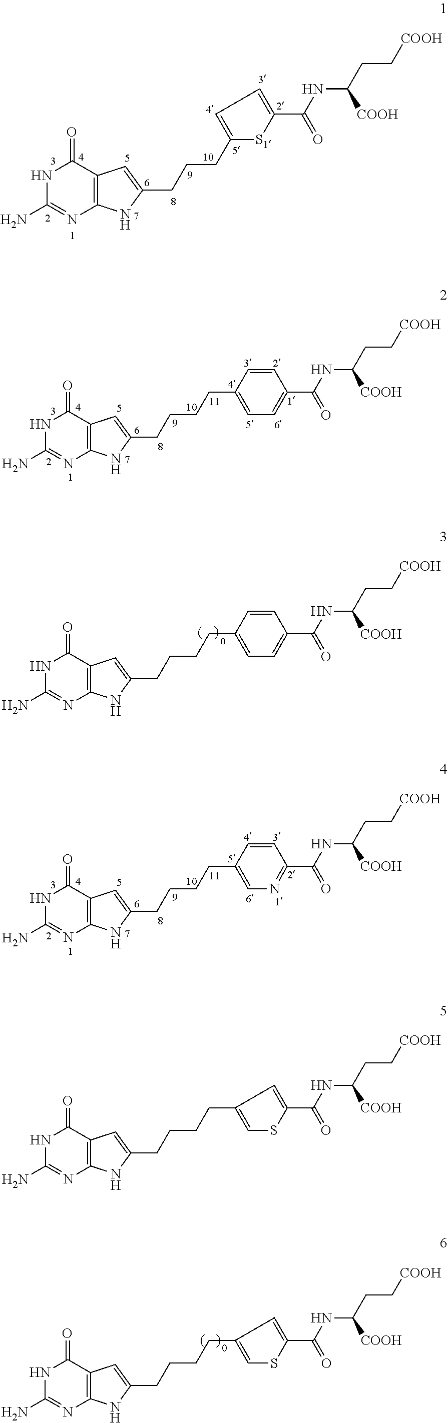

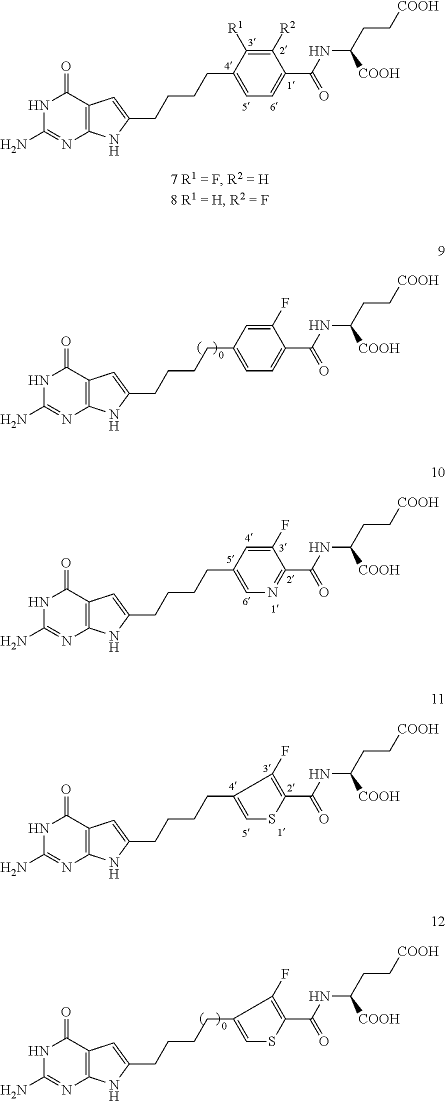

In certain preferred embodiments of this method, the compound has the structure 3, 11, or 12, above. In certain other embodiments of this method, the M2-like macrophage is an M2-like FRβ-expressing macrophage, including for example, wherein the M2-like FRβ-expressing macrophage is a tumor-associated macrophage (TAM).

In other embodiments of this invention, the method includes wherein the administration of the compound results in anti-tumor efficacy accompanied by decreased M2-like FRβ-expressing macrophages and increased CD3+ T cells, and wherein CD4+ and CD8+ T cells are unaffected.

In other embodiments, the method includes effecting anti-tumor efficacy of the compound in the therapy of epithelial ovarian cancer (EOC) in an intact immune system and targeting an immunosuppressive tumor microenvironment (TME).

In another embodiment of this invention, a method of inhibiting M2-like macrophages in a patient is provided comprising administering to a patient having ovarian cancer a therapeutically effective amount of a composition comprising a compound that is a substituted pyrrolo[2,3-d]pyrimidine antifolate inhibitor having selective efficacy to FRβ expressing tumor cells, and an acceptable pharmaceutical carrier. The method includes wherein the substituted pyrrolo[2,3-d]pyrimidine antifolate inhibitor is a compound selected from the group consisting of the following structures 1-12:

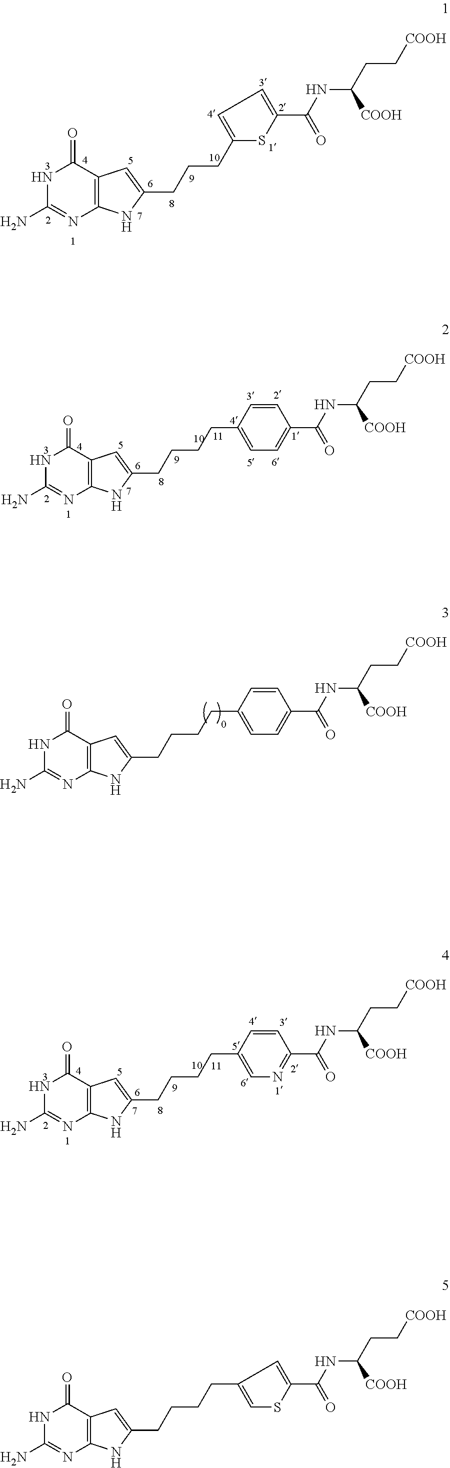

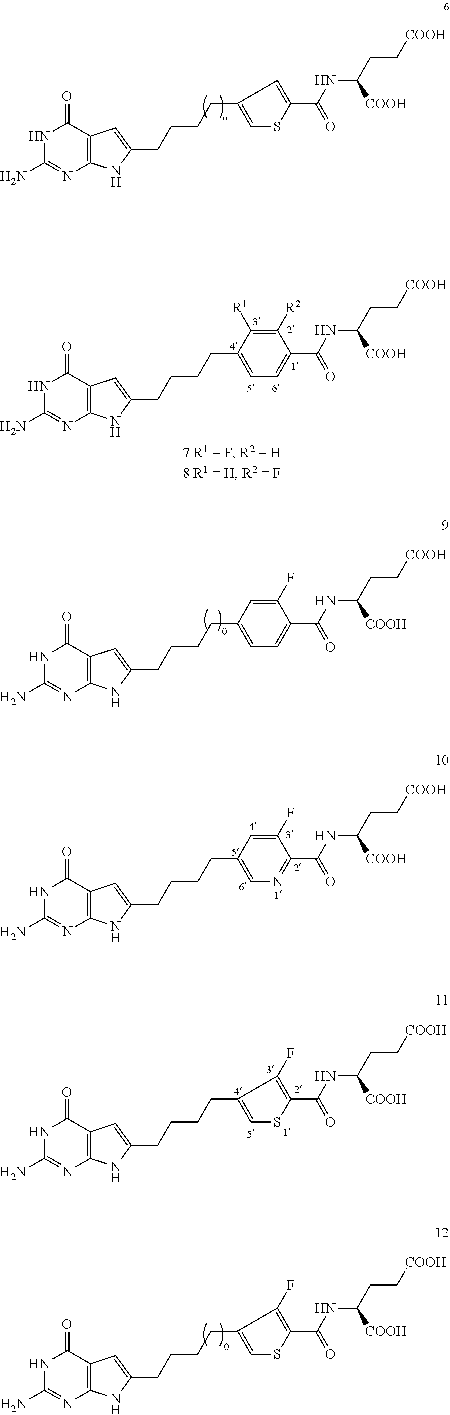

The method includes wherein the acceptable pharmaceutical carrier is one selected from the group of saline, dextrose and water, and sucrose. The method further includes wherein the composition includes one or more pharmaceutically acceptable excipients, fillers, binders, and surfactants. The method includes effecting anti-tumor efficacy of the composition in the therapy of epithelial ovarian cancer (EOC) in an intact immune system and targeting an immunosuppressive tumor microenvironment (TME).

In another embodiment of this invention, a method of inhibiting M2-like macrophages in a patient is provided comprising administering to a patient having ovarian cancer a therapeutically effective amount of a FRβ-transported C1 inhibitor compound having selective efficacy to FRB expressing tumor cells. This method includes wherein the FRβ-transported C1 inhibitor compound is a substituted pyrrolo[2,3-d]pyrimidine antifolate inhibitor compound having selective efficacy to FRβ expressing tumor cells. The method includes wherein the substituted pyrrolo[2,3-d]pyrimidine compound is a compound selected from the group consisting of the following structures 1-12:

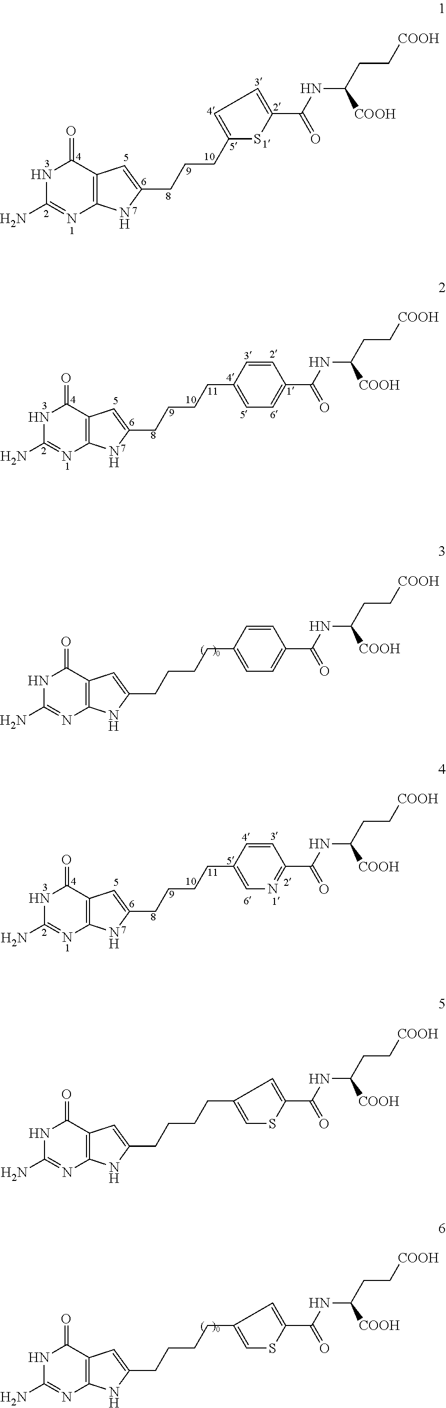

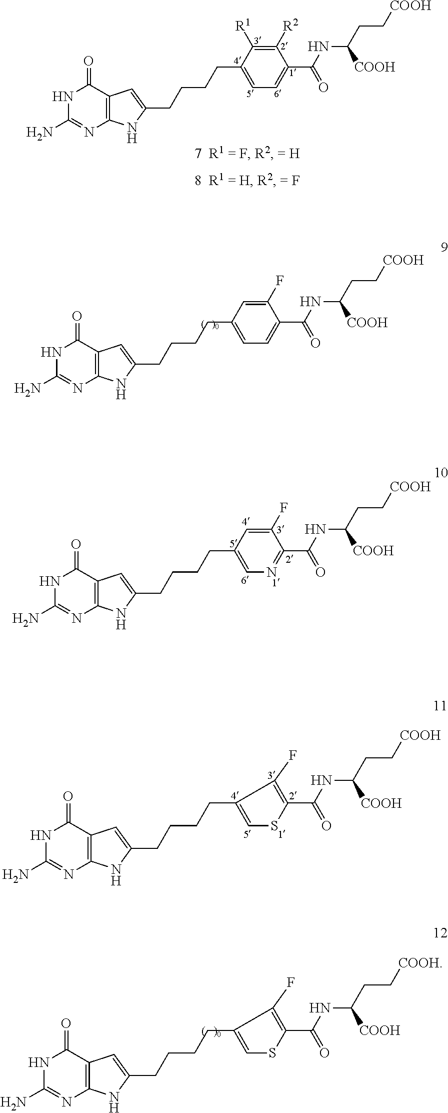

This method includes effecting anti-tumor efficacy of said compound in the therapy of epithelial ovarian cancer (EOC) in an intact immune system and targeting an immunosuppressive tumor microenvironment (TME).

Another embodiment of this invention provides a method of inhibiting M2-like macrophages in a patient comprising administering to a patient having ovarian cancer a therapeutically effective amount of a composition comprising a FRβ-transported C1 inhibitor compound having selective efficacy to FRβ expressing tumor cells and a pharmaceutically acceptable carrier. The method includes wherein the pharmaceutically acceptable carrier is one selected from the group of saline, dextrose and water, and sucrose. This method further includes wherein the composition includes one or more pharmaceutically acceptable excipients, fillers, binders, and surfactants.

In certain other embodiments of this invention, a FRβ-transported C1 inhibitor compound having selective efficacy to FRβ expressing tumor cells for use in inhibiting M2-like macrophages for treating a patient having ovarian cancer, is provided. The FRβ-transported C1 inhibitor compound having selective efficacy to FRβ expressing tumor cells is selected from the group consisting of substituted pyrrolo[2,3-d]pyrimidine antifolate inhibitor compounds having selective efficacy to FRβ expressing tumor cells. The FRβ-transported C1 inhibitor compound having selective efficacy to FRβ expressing tumor cells that is a substituted pyrrolo[2,3-d]pyrimidine antifolate inhibitor compound is selected from the group consisting of the following structures 1-12:

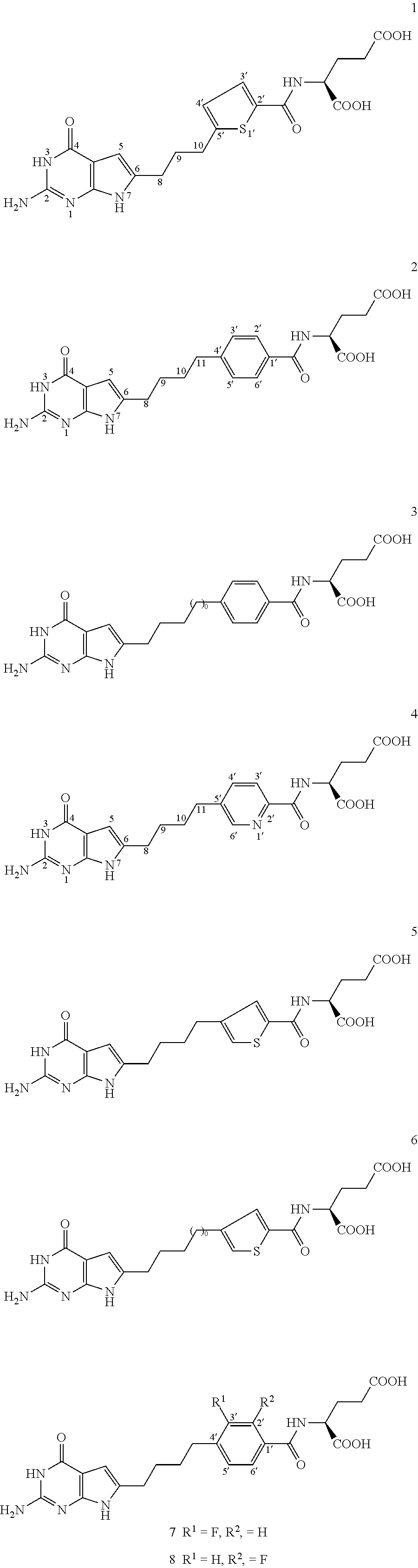

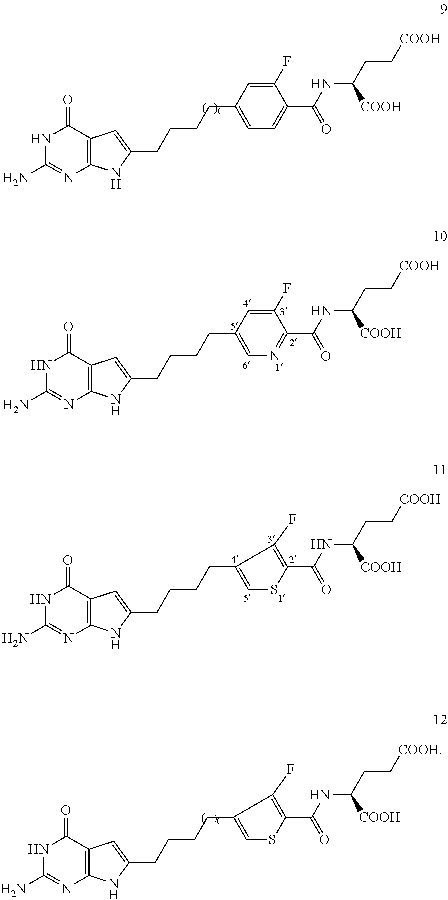

### Results

### Expression of Folate Transporters and Folate-Dependent Enzymes in De Novo Purine Biosynthesis in BR-5 and Br-Luc EOC Cells, Compared to Human EOC Cell Lines and Primary EOC and Ovary Specimens.

We discovered novel 6-substituted pyrrolo[2,3-d]pyrimidine compounds (AGF94, AGF278, AGF283, see FIG. 1 and Schematic 1) that inhibit de novo purine biosynthesis at GARFTase and target a range of human tumor cells including EOC via selective transport by FRs and PCFT over RFC^{6, 15, 16, 19, 20}. Potent in vivo efficacy by this series was demonstrated in human EOC xenografts in a severe combined immunodeficient (SCID) mouse model^{6, 15}. However, anti-tumor efficacy against a syngeneic mouse model of HGSOC including the potential role of the TME in treatment response has not been tested. Thus, we expanded our studies of these C1 inhibitors to include a FVB-syngeneic mouse model of EOC, BR-Luc (p53−/−; Brca−/−; myc; Akt), derived from the BR-5 cells^{17, 18}.

We previously reported substantial expression of FRa and PCFT transcripts in HGSOC from patients⁶. FRa transcripts were significantly elevated in HGSOC over normal ovary and increased with disease stage, whereas PCFT transcripts were more modestly increased over normal ovary and were independent of stage⁶. FRa and PCFT levels in EOC cell lines (i.e., SKOV3, IGROV1) were similar to those in the patient EOC specimens⁶.

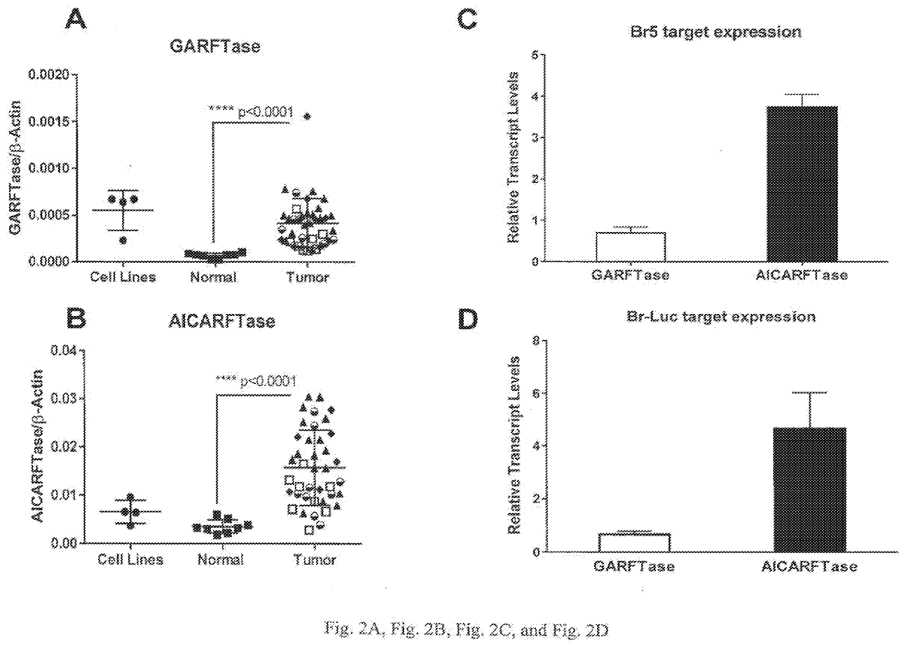

We initially analyzed gene expression of the folate-dependent de novo purine biosynthetic enzymes GARFTase and AICAR formyl transferase (AICARFTase) in primary EOCs. We used a separate panel of cDNAs from before⁶, including normal ovary (n=8) and HGSOC (n=39; 8 stage I, 9 stage II, 17 stage III, 6 stage IV) specimens (see Table S2) to measure expression of GARFTase and AICARFTase. Transcript levels for the EOC primary specimens were compared to those in the EOC cell lines IGROV1, SKOV3, A2780 and A2780 E-80. Transcripts for these enzyme targets in EOC cell lines overlapped with those for primary EOC specimens; transcript levels in primary EOC specimens were substantially increased over those in normal ovary (median 5.2-fold for GARFTase; median 4.4-fold for AICARFTase) (FIGS. 2A-B), suggesting the importance of this key anabolic pathway to HGS EOC.

For comparison, we profiled the relative expression of GARFTase and AICARFTase in the mouse models of HGSOC, BR-5 and BR-Luc. High levels of GARFTase and AICARFTase were measured in BR-5 and BR-Luc cells (increased ˜1.5- and ˜4-fold, respectively, compared to mouse liver) (FIGS. 2C-D).

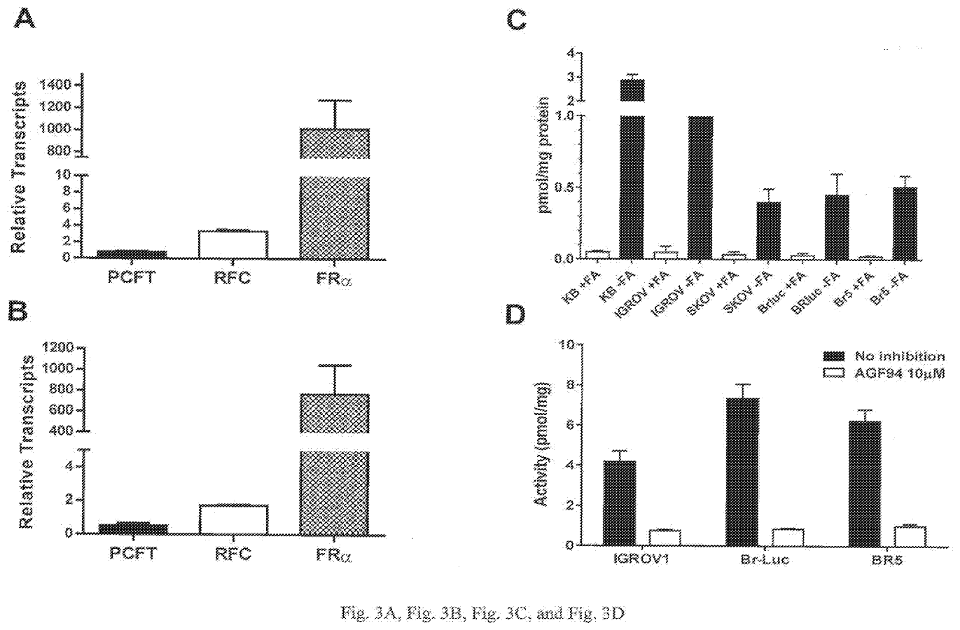

We characterized BR-5 and BR-Luc murine EOC cells for expression of the major folate transporters, FRa, PCFT and RFC. The levels of PCFT and RFC transcripts in BR-5 and BR-Luc cells were substantial, with PCFT at ˜40% and ˜60%, respectively, of those in normal mouse liver; RFC transcripts were ˜2- and ˜3-fold increased over levels measured in mouse liver (FIG. 3A-B). FRa transcripts in BR-5 and BR-LUC cells were highly elevated and were increased 763- and 1015-fold, respectively, over the level of FRa in liver.

We used [3H]folic acid binding to surface FRs in BR-5 and BR-Luc EOC cells, as a functional readout for FRs and to compare FR protein levels in the murine EOCs to those for human tumor cell lines including FRa-expressing KB cells, and SKOV3 and IGROV1 EOC cells⁶. [³H]Folic acid cell surface binding was measured in the presence and absence of excess (10 μmol/L) non-radioactive folic acid to demonstrate binding specificity^{6, 21}. Total FR levels by this method were in rank order KB>IGROV1>SKOV3˜BR-Luc˜BR-5 (FIG. 3C). Thus, FRa levels in BR-5 and BR-Luc murine cells approximate those in the cisplatin resistant SKOV3 human EOC cells and by extension primary EOC specimens from patients⁶.

To functionally assess PCFT levels, PCFT transport activity was measured in the BR-5 and BR-Luc cells with 0.5 μmol/L [³H]methotrexate (MTX) over 5 minutes at pH 5.5, corresponding to the optimal pH for transport by this system^{6, 22, 23}. Under these conditions, uptake by FRs is nominal⁶. To demonstrate specificity for transport by PCFT, we added excess (10 μM) non-radioactive AGF94 in parallel incubations which nearly completely ablated uptake as a competitive inhibitor^{6, 15}. Results were compared to those in IGROV1 human EOC cells which express abundant PCFT along with FRa, analogous to primary patient EOCs⁶. As shown in FIG. 3D, substantial PCFT transport activity was detected in BR-5 and BR-Luc cells, with slightly increased transport in the murine EOC cells compared to human IGROV1 cells.

Collectively, these results establish that the key determinants of sensitivity to the pyrrolo[2,3-d]pyrimidine antifolates in the BR-5/BR-Luc murine EOC models closely approximate those of the human EOC cell lines and primary EOC specimens. They strongly suggest that, as with human EOCs, the murine HGSOC cells should be vulnerable to the inhibitory effects the pyrrolo[2,3-d]pyrimidine antifolates.

### In Vitro Efficacy of C1 Inhibitors for BR-5 and BR-Luc Murine HGSOC Cells.

We previously established an extensive structure-activity-relationship (SAR) profile for 6-substituted pyrrolo[2,3-d]pyrmidine compounds with modifications in the side chain, including the bridge length and the nature of the aromatic moiety, as selective transport substrates for FRs and PCFT, and as inhibitors of de novo purine nucleotide biosynthesis^{7, 22, 24} To further characterize the murine HGSOC models BR-5 and BR-Luc, we tested the in vitro antiproliferative activities of our lead C1 pyrrolopyrimidine inhibitors AGF94, AGF278 and AGF283^{15, 16} (FIG. 1 and Schematic 1). Results with BR-5 and BR-Luc cells were compared to those for IGROV1 human EOC cells (Table 1) in the absence and presence of excess folic acid (200 nM), which selectively blocks FR-mediated drug uptake without effects on RFC or PCFT⁶. BR-5 and BR-Luc cells showed potent inhibition by all the compounds with IC₅₀ values ranging from ˜6 nM for AGF94 to ˜75 nM for AGF283 (both with BR-Luc cells) (Table 1). Further, inhibitions of the murine EOCs by all compounds approximated those for IGROV1 human EOC cells. For both murine and human EOC cells, inhibitions were all significantly decreased by excess folic acid, establishing their cellular uptake by FRa with secondary uptake by PCFT^{6, 25}.

In Vivo Analysis of Efficacy for AGF94 with Intraperitoneal BR-Luc EOC.

An important goal was to extend our prior experience in therapeutic studies of pyrrolo[2,3-d]pyrimidine inhibitors which utilized subcutaneous (SC) human EOC xenografts in an immune-compromised SCID mouse model^{6, 16} to evaluation of a syngeneic FBV mouse model of HGSOC. We selected the lead compound AGF94 for these studies and chose the BR-Luc EOC model for further studies based on its in vitro sensitivity to AGF94 which paralleled that for IGROV1 human EOC cells (Table 1). Further, the BR-Luc tumor can be imaged by luminescence imaging.

As proof-of-concept antitumor efficacy of AGF94 in mice with intact immune function and to recapitulate the clinical-pathologic features of HGSOC, we initially used intraperitoneal (IP) engraftment of BR-Luc tumors in female FVB mice. IP presentation of the BR-Luc tumor allowed for dissemination throughout the peritoneum, as occurs in patients.

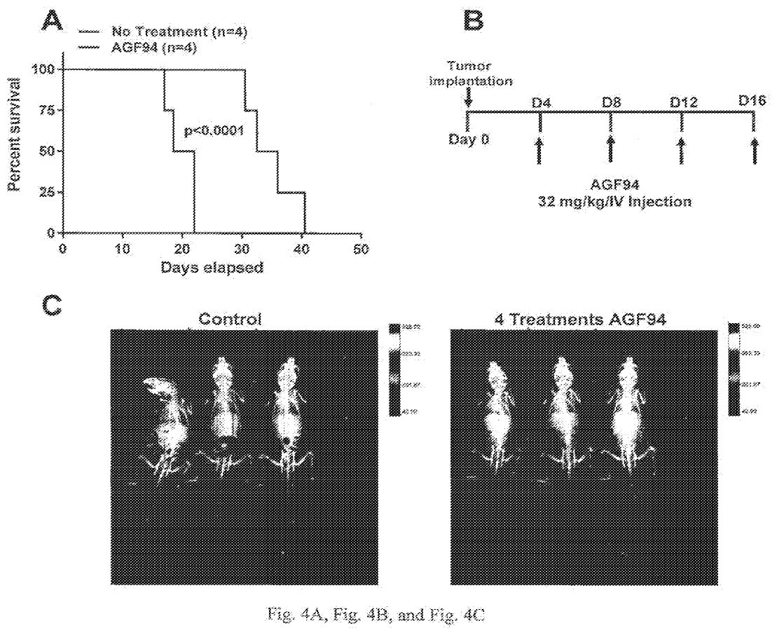

For all in vivo studies, mice were maintained on a low-folate diet to reduce highly elevated serum folate concentrations (from the standard folate-replete diet) to levels approximating those in humans^{6, 15, 16}. BR-Luc cells (5×10⁶/mouse) were injected IP on day 0, and the mice were non-selectively distributed to each arm (control and AGF94-treated (n=4), with separate matching cohorts for imaging (n=3)). AGF94 was administered IV (Q4dx4 at 32 mg/kg/injection) beginning on day 4, for a total dose of 128 mg/kg (FIG. 4B). AGF94-treated mice sustained a median 5.5% body weight loss nadir on days 18 and 26 with full recovery on day 28. Disease progression and anti-tumor efficacy were monitored by weighing mice daily, palpating and measuring IP tumor masses, observing overt symptoms and luminescence imaging. Mice were euthanized at disease end point, characterized by abdominal distension and onset of labored breathing due to ascites accumulation (>1-2 mL), and/or the presence of palpable tumor mass(es)>5% body weight (i.e., up to 1 g of cumulative solid tumor burden). Upon necropsy, accumulation of hemorrhagic ascites (1-3 mL) was observed, accompanied by disseminated metastatic disease, involving numerous small tumor nodes which homed to the ovaries, mesenteric lymph nodes and adipose tissue associated with the pancreas and GI space.

AGF94 was active against the BR-Luc orthotopic IP model (FIGS. 4A and C). Following 4 treatments with AGF94, tumors were undetectable by luminescence imaging (FIG. 4C shows imaging of a matching parallel treated cohort including 3 mice in each arm) with AGF94), although the tumors regrew following cessation of therapy. The median days to death in the control mice were 20.25 days (range 17.5-22 days) compared to 34.25 days (range 30.5-40.5 days) for mice treated with AGF94; this yields a 70% increased lifespan (% ILS) and 2.1 logs of gross cell kill based on a 2 day doubling time for IP implanted cells.

### In Vivo Anti-Tumor Efficacy Analysis of AGF94 Against Late-Stage BR-Luc and Impact on Infiltrating Immune Microenvironment.

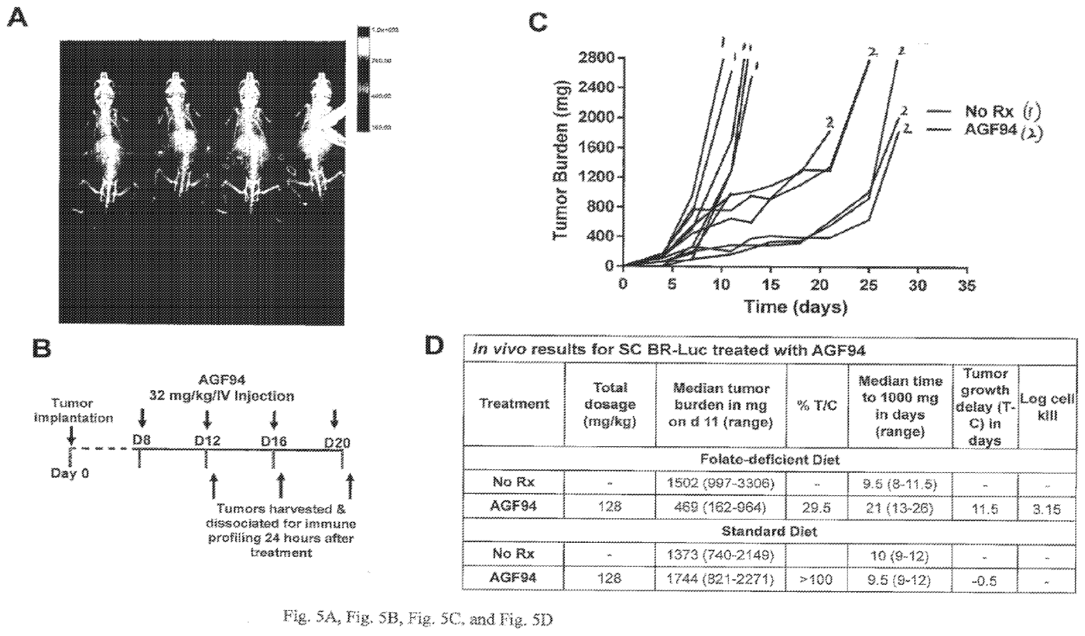

We extended our initial in vivo studies with AGF94 to advanced-stage SC implanted BR-Luc in FVB mice which permits careful monitoring of the relationships between anti-tumor efficacy and the impact of drug treatment on the infiltrating immune microenvironment without considerations of metastatic spread. FVB mice were maintained on a folate-deficient diet and on day 0 (FIG. 4B) the mice were implanted bilaterally SC with BR-Luc tumor fragments by trocar. On day 7, the tumor sizes were measured and the mice were non-selectively distributed into various treatment and control groups (6 mice/group) for determinations of antitumor efficacy with parallel cohorts to accommodate imaging and tumor immune infiltration. The median tumor burden on day 7 for BR-Luc advanced stage disease was 388 mg (range 356-412 mg). Matching control and treated cohorts (n=4 mice group) were maintained on a standard (folate replete) mouse diet (day 7 median tumor burden was 218 mg (range 213-223 mg). On day 8, drug treatment was initiated and the mice were dosed with AGF94 (32 mg/kg IV on a Q4dx4 schedule). FIG. 5B summarizes the treatment scheme and FIG. 5A shows the luminescence detection of BR-Luc tumors on day 7 before initiation of AGF94 treatment. Tumor growth was monitored twice weekly and all mice were observed and weighed daily for the duration of the study.

For mice maintained on the folate-deficient diet, antitumor activity as assessed by the T/C value on day 11 (3 days post first treatment with AGF94) was a median 31.2% of the control (see table in FIG. 5D) (further determinations of % T/C values were not possible due to euthanization of controls reaching tumor burden endpoints on or after 11 days; Td=1.1 days). For quantitative assessments of anti-tumor efficacy, tumor growth delay (T-C) was employed (defined as the median time for the tumor to reach 1 g for treated (T) and control (C) groups. AGF94-treated mice yielded a median 11.5 day tumor growth delay and 3.15 logs of gross cell kill (defined as T-C/3.32×Td). Again, AGF94 treatment was well tolerated with the only dose-limiting symptom being weight loss (nadir of 6.4% on day 18 and full recovery by day 26). For the mice maintained on the standard diet, AGF94 was inactive as expected (% T/C>100%, and no log kill; there was no weight loss and there were no other adverse symptoms (Table 5D)).

An important goal of our study was to explore the impact of 2, 3 or 4 doses of AGF94 upon the tumor microenvironment, including infiltrating lymphocytes accompanying its anti-tumor effects. Further, since AGF94 is transported in part by FRs, including FRβ¹⁶, we measured its effects on the FRβ-expressing TAMs based on published reports that targeting macrophages via FRβ may have therapeutic potential for treating inflammatory diseases and cancer^{13, 14, 26}.

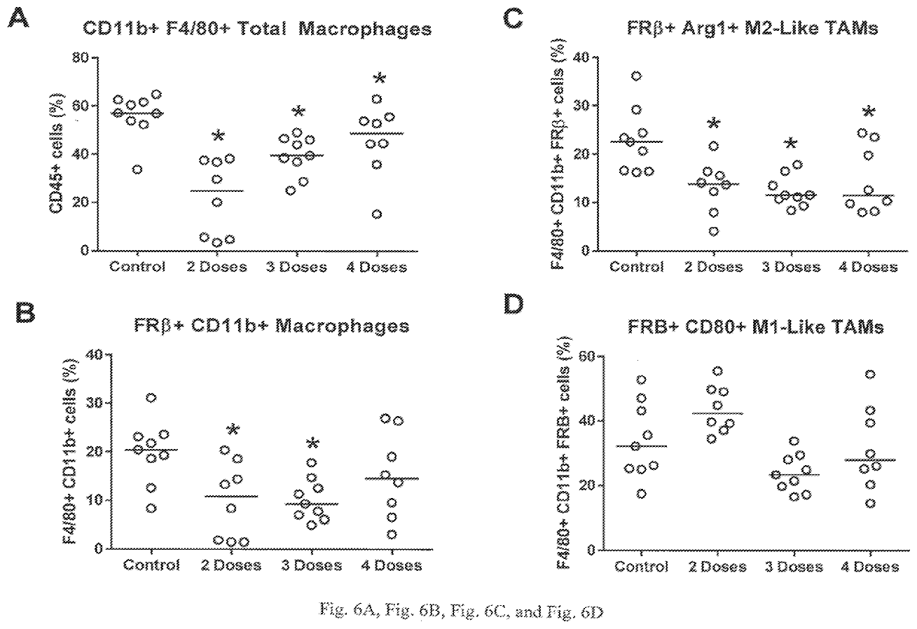

To assess the effects of drug treatment upon the infiltrating immune population, tumors and spleens (as a control) were harvested from parallel cohorts of AGF94-treated mice after 2, 3 and 4 injections. The impact of drug treatment was initially determined on the total macrophage population, defined as dual expressing CD11b+ and F4/80+ cells from live CD45+ cells including the FRβ-expressing population (FIGS. 6A and 6B). After treatment with AGF94, there were statistically significant decreases after 2 or 3 doses on the CD11b+/F4/80+/FRβ+ population which appeared to diminish with subsequent dosing.

We extended our analysis to include the M1-like macrophage population defined as FRβ+, CD80+ TAMs and the M2-like macrophage population defined as FRβ+, Arg1+ TAMs. AGF94 exerted at most a modest impact on the M1-like TAMs from 2-4 doses (FIG. 6D); however, there was a consistent and highly significant decrease in the M2-like TAMs (FIG. 6C) that generally paralleled the observed changes in the CD11b+/F4/80+/FRβ TAM population (FIG. 6B). Thus, our results suggest that that AGF94 can target M2-like TAMs that express FRβ14.

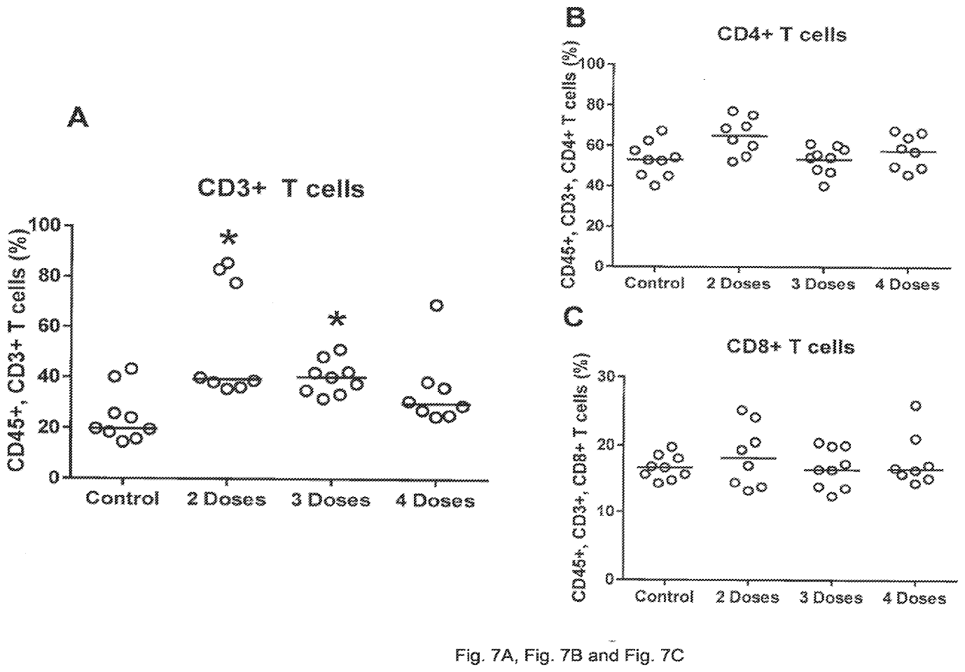

We also examined the impact of AGF94 dosing on the infiltrating T cell populations. Treatment with AGF94 was accompanied by a statistically significant increase (˜50-100%) in the CD3+ T cells, as defined by the percentage of live CD45+ cells (FIG. 7A). There was no impact on the proportions of CD4+ and CD8+ T cells in the tumors (FIGS. 7B-C) and spleens (not shown) (measured as a percentage of CD3+ T cells) after 4 doses of AGF94.

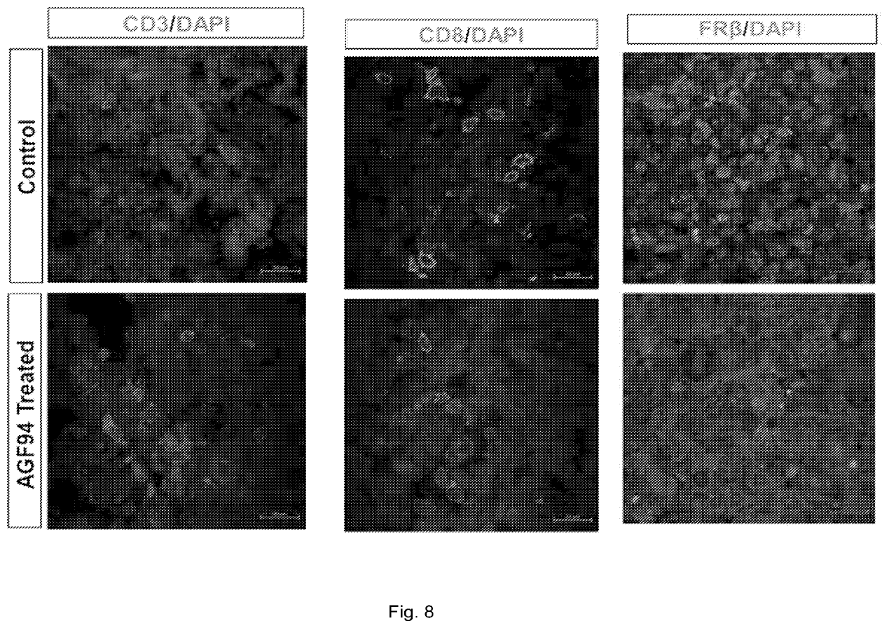

As validation of our flow cytometry results and to assess the spatial landscape of the immune infiltrate, we performed immunofluorescence (IF) on tumor sections from control and drug-treated mice following 4 doses of AGF94 (FIG. 8). Both CD3+ and CD8+ T cells were detected in sections from AGF94-treated mice, albeit to variable extents, consistent with the flow cytometry results. Importantly, T cells were disseminated throughout the tumor bed regardless of treatment status, substantiating the quantitative flow cytometry results which suggest these cells are not diminished with AGF94. As expected, FRβ positive cells were most easily detected in the sections from untreated mice, with very few FRβ positive cells in the AGF94-treated sections (FIG. 8).

### DISCUSSION

AGF94 is a prototype FR- and PCFT-targeted pyrrolo[2,3-d]pyrimidine antifolate previously reported to show broad-ranging anti-tumor efficacy (including effectiveness against human EOC xenograft models)^{6, 15, 19, 20}. AGF94 is potent inhibitor of GARFTase^{16, 27}, the first folate-dependent step in de novo purine biosynthesis, a critical anabolic pathway in malignant cells²⁸. Inhibition of purine nucleotide biosynthesis kills tumors independent of the wild-type/mutant p53 status^{29, 30}, shows tumor selectivity based on impaired purine salvage^{31, 32}, and results in suppression of mTOR signaling^{33, 34}. The tumor microenvironment contains a host of infiltrating immune cells, including TAMs and T-lymphocytes, with TAMs considered the principal immune cellular component which results in an immunosuppressive environment. FRB is expressed on IL-10-producing M2-like macrophages (CD163+, CD68+, CD14+IL-10), corresponding to the anti-inflammatory/pro-tumor TAM subtype, prompting substantial interest in depleting TAMs by exploiting FRβ on the surface of macrophages. A BIM (BCL-2-interacting mediator of cell death) plasmid encapsulated in a folate “lipoplex” was developed to target the tumor microenvironment in lung cancer and an anti-mouse FRβ monoclonal antibody conjugated to Pseudomonas exotoxin A depleted TAMs and reduced tumor growth a C6 rat glioma model.

Further, a folate-conjugated TLR7 agonist showed in vivo activity in assorted tumor models and reversed expression of a high M2-like to M1-like macrophage ratio and increased the infiltration of cytotoxic CD8 T cells. The present method describes the novel pyrrolo[2,3-d]pyrimidine antifolate AGF94 for dual targeting HGSOC directly, as well as indirectly via its effects on the tumor microenvironment. AGF94 is a prototype FR- and PCFT-targeted pyrrolo[2,3-d]pyrimidine antifolate previously reported to show broad-ranging anti-tumor efficacy (including human EOC xenograft models). AGF94 is potent inhibitor of G ARFTase, the first folate-dependent step in de novo purine biosynthesis, a critical anabolic pathway in malignant cells. Inhibition of purine nucleotide biosynthesis kills tumors independent of the wild-type/mutant p53 status, shows tumor selectivity based on impaired purine salvage, and results in suppression of mTOR signaling.

In this method, we used a syngeneic immunocompetent FVB mouse model of orthotopic HGSOC Br-Luc that accurately recapitulates the histology and progression of human HGSOC^{17, 18} and found that AGF94 effected potent in vivo anti-tumor efficacy. Our results with an orthotopic IP BR-Luc EOC model were extended to an advanced stage SC model of BR-Luc so as to better study the changes in the TME immune populations accompanying treatments with AGF94. Anti-tumor activity was accompanied by a direct impact on the TME including significantly decreased FRβ-expressing TAMs, with no evidence of CD3+ T cell depletion or impact on the relative proportions of CD4+ and CD8+ T cells after drug treatment.

Our finding of decreased M2-like TAMs accompanying treatment with AGF94 is highly significant, as FRβ was implicated as a marker of immunosuppressive M2-like macrophages^{13, 14, 26} and a decreased M2-like TAMs thus results in reduced tumor burden. However, no candidate drug has yet to advance to the clinic. The lack of CD3+ T cell reduction suggests that there may also be potential for combination therapies to enhance anti-tumor T cell activity upon treatment with AGF94. Based on these results, we set forth that in this invention that FRβ-transported C1 inhibitor compounds such as the substituted pyrrolo[2,3-d]pyrimidines set forth in Schematic 1 including, for example but not limited to AGF94, represent an exciting new approach for therapy of HGSOC in a patient through its ability to directly target the tumor via uptake by FRa and PCFT, and its effects on the TME, particularly FRβ-expressing TAMs.

Interestingly, S-adenosyl methionine (SAM) depletion was reported to inhibit M1-like macrophages³⁵, raising the possibility that C1 metabolism is indeed critical for both M1-like and M2-like macrophage populations alike. This invention provides evidence that FRβ is important for targeting TAMs in EOC and that the present method of inhibiting cancer-associated TAMS in cancer patients by administering a therapeutically effective amount of a pyrrolo[2,3-d]pyrimidine antifolate inhibitor that is selective for FRβ (folate receptor beta) is a targeted therapy for high grade serous ovarian cancer (HGSOC). The present method inhibits tumor associated macrophages by use of FRβ-transported C1 inhibitor compounds such as for example but not limited to pyrrolo[2,3-d]pyrimidine compounds having FRβ selectivity.

FIG. 1 shows examples of structures of 6-substituted pyrrolo[2,3-d]pyrmidine inhibitors of de novo purine nucleotide biosynthesis used in the methods of this invention. Structures are shown for AGF94, AGF278, and AGF283, selective substrates for FRs and PCFT over RFC, and inhibitors of the folate-dependent purine biosynthetic enzyme, GARFTase^{15, 16}.

FIG. 2A shows expression of GARFTase transcripts in primary epithelial ovarian cancer (EOC) patient samples. FIG. 2B shows expression of AICARFTase transcripts in primary EOC patient samples. FIG. 2C shows transcript levels of cytosolic GARFTase and AICARFTase C1 metabolic targets in the BR-5 syngeneic mouse models of HGSOC by real-time RT-PCR. FIG. 2D shows transcript levels of cytosolic GARFTase and AICARFTase C1 metabolic targets in the Br-Luc syngeneic mouse models of HGSOC by real-time RT-PCR. FIGS. 2A and 2B show expression of GARFTase and AICARFTase transcripts in primary EOC patient samples. Transcript levels for GARFTase (FIG. 2A) and AICARFTase (FIG. 2B) were measured using cDNAs from primary specimens including normal ovary (n=8) and HGSOC (n=39) (OriGene) and results were compared to those for EOC cell lines including IGROV1, SKOV3, A2780 and A2780 E-80. Transcript levels were normalized to β-actin transcripts. Statistical analyses were performed between normal samples/tissues and tumor samples/tissues using the Wilcoxon rank sum test. FIGS. 2C and D show transcript levels of cytosolic (GARFTase and AICARFTase) C1 metabolic targets were determined in the BR-5 (FIG. 2C) and BR-Luc (FIG. 2D) syngeneic mouse models of HGSOC by real-time RT-PCR. Transcripts were normalized to β-actin transcripts and results are shown relative to levels in mouse liver (assigned a value of 1). Results are presented as mean values±standard errors from at least three experiments. P values are as follows: **** p<=0.0001; *** p<=0.001; p<=0.01; p<=0.05. See Table 2 for patient characteristics and pathology.

FIG. 3A shows folate transporter expression in BR-Luc syngeneic HGSOC mouse models. FIG. 3B shows folate transporter expression in BR-5 syngeneic HGSOC mouse models. FIG. 3C shows total surface FRa that was measured by titration with [3H]folic acid at 0° C. with and without unlabeled 10 μmol/L non-radioactive folic acid. FIG. 3D shows PCFT uptake that was assayed using [3H]MTX (0.5 μM) at pH 5.5 at 37° C. in the absence and presence of 10 μmol/L non-radiolabeled AGF94. FIGS. 3A and B show folate transporter expression in BR-5 and BR-Luc syngeneic HGSOC mouse models. Transcript levels for PCFT, RFC and FRa were measured by real-time RT-PCR for BR-Luc (FIG. 3A) and BR-5 (FIG. 3B). Transcript levels are presented as mean values±standard errors from at least three experiments. Transcripts were normalized to β-actin transcripts and levels are shown relative to those in mouse liver (assigned a value of 1). FIG. 3C shows the total surface FRa that was measured by titration with [3H]folic acid at 0° C. with and without unlabeled 10 μmol/L non-radioactive folic acid. Results are presented at mean values±standard errors from at least three experiments. FIG. 3D shows PCFT uptake that was assayed using [3H]MTX (0.5 μM) at pH 5.5 at 37° C. in the absence and presence of 10 μmol/L non-radiolabeled AGF94, as previously described¹⁶. Results are expressed as average values (+/−range) from at least 2 experiments.

FIG. 4A shows AGF94 efficacy trial overall survival in IP BR-Luc model. FIG. 4B shows a treatment scheme for the AGF94 efficacy trial of FIG. 4A. FIG. 4C shows luminescent images for IP BR-Luc tumors in FVB mice (right panel of FIG. 4C) 1 day following treatment with AGF94 (32 mg/kg×4 doses) and control mice (left panel of FIG. 4C). Overall survival (FIG. 4A) and luminescent images (FIG. 4C) are shown for IP BR-Luc tumors in FVB mice 1 day following treatment with AGF94 (32 mg/kg×4 doses). The treatment scheme (FIG. 4B) is also shown. For control mice, a median of 22 days was measured compared to a median of 33 days for the AGF94-treated mice. Statistical analysis was performed using the log-rank (Mantel-Cox) test. A p value of <0.0001 was calculated. Luminescent images were collected over 3 min for both control and AGF94-treated mice (4 doses with 32 mg/kg) and overlayed on top of an x-ray image.

FIG. 5A shows luminescent images of AGF94 efficacy in a subcutaneous BR-Luc model. FIG. 5B shows a trial design schematic for the AGF94 efficacy trial of FIG. 5A. FIG. 5C shows plotted results for the BR-Luc trial efficacy arm with AGF94 by individual mice. FIG. 5D shows a table that summarizes the results of the in vivo trial with SC BR-Luc xenografts treated with AGF94 for mice maintained on both the folate-deficient and folate replete diets. Luminescent images were collected over 3 min and overlayed on top of an x-ray image. The image was obtained 7 days after the tumor was allografted and 1 day prior to treatment initiation. The trial design schematic is shown (FIG. 5B). BR-Luc tumors were engrafted SC bilaterally; treatment with AGF94 began after 8 days when the tumors were palpable (˜400 mg). Tumors were harvested, dissociated and flow cytometry was performed 24 h after 2, 3, and 4 doses of AGF94. (C) Results are plotted for the BR-Luc trial efficacy arm with AGF94 by individual mice. Female FVB mice were implanted bilaterally SC with BR-Luc tumors and AGF94 treatment was initiated on day 8 following tumor implantation. AGF94 was dosed as Q4dx4 at 32 mg/kg/IV injection. T/C % was determined on day 11. FIG. 5D sets forth a table that summarizes the results of the in vivo trial with SC BR-Luc xenografts treated with AGF94 for mice maintained on both the folate-deficient and folate replete diets.

FIG. 6A shows the impact of AGF94 treatment on tumor infiltrating macrophages, namely the percentage of CD11b+ and F4/80+ macrophages. FIG. 6B shows the percentage of FRβ+, gated off CD11b+ and F4/80+ macrophages following 2, 3, or 4 doses of AGF94. FIG. 6C shows the percentage of Arg1+FRβ+, gated off CD11b+ and F4/80+ macrophages, following 2, 3 or 4 doses of AGF94. FIG. 6D shows the percentages of CD80+FRβ+, gated off CD11b+ and F4/80+ macrophages following 2, 3, or 4 doses of AGF94. To assess the impact on the immune populations in mice treated with AGF94, the immune populations from tumors and spleens were harvested after 2, 3, and 4 treatments. Results are shown for the percentage of CD11b+ and F4/80+ macrophages (FIG. 6A), and percentage of FRβ+, gated off CD11b+ and F4/80+ macrophages (FIG. 6B) with 2, 3, or 4 doses of AGF94. Percentage of Arg1+FRβ+, gated off CD11b+ and F4/80+ macrophages are shown in FIG. 6C following 2, 3 or 4 doses of AGF94. Percentages of CD80+FRβ+, gated off CD11b+ and F4/80+ macrophages are shown (FIG. 6D) following 2, 3, or 4 doses of AGF94. Results are presented as mean values±standard errors from at least six mice per group. Statistical comparisons were made between AGF94-treated and control mice. * p<0.05, NS=not significant.

FIG. 7A shows the impact of AGF94 treatment on tumor infiltrating T cells, namely, percentages of CD3+ T cells from single cell suspensions of tumor and infiltrating lymphocytes following 2, 3 and 4 doses of AGF94. FIG. 7B shows percentage of CD4+ T cells are shown from single cell suspensions of tumor and infiltrating lymphocytes following 2, 3 and 4 doses of AGF94. FIG. 7C shows percentage of CD8+ T cells are shown from single cell suspensions of tumor and infiltrating lymphocytes following 2, 3 and 4 doses of AGF94. The CD4+ and CD8+ populations were gated off CD45+ and CD3+ cells. Results are presented as mean values±standard errors from at least six mice per group. Statistical comparisons were made between AGF94-treated and control mice. * p<0.05, NS=not significant.

FIG. 8 shows immunofluorescence staining of immune populations. FIG. 8 top panels are controls. FIG. 8 bottom panels are AGF94 treated mice tumors with the immunofluorescence staining for tumor sections stained with CD3 (FIG. 8 bottom left panel), CD8 (FIG. 8 bottom middle panel), or FRβ (red) antibodies (FIG. 8 bottom right panel) with DAPI counterstain (blue) (scale bars, 20 μm). Staining is shown for representative tumor sections from control and AGF94-treated mice.

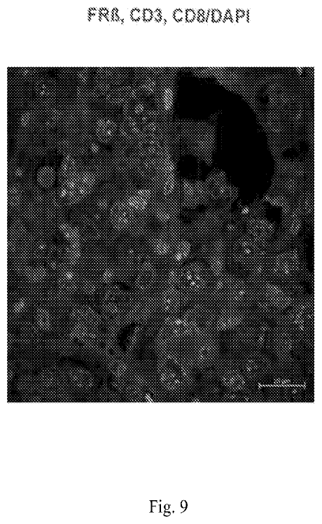

FIG. 9 shows negative control for immunofluorescence. For immunofluorescence staining shown in FIG. 8, the secondary antibody was used (anti-Rabbit IgG (H+L) Alexa Fluor 647 (1:200, Thermo Fisher, Catalog #A-21245) to show specificity for CD3, CD8, and FRβ. Scale bar set to 20 μm.

### Materials and Methods

Reagents-All chemicals were obtained in the highest available purities from commercial sources. Leucovorin [(6R,S) 5-formyl-THF] and MTX were provided by the National Cancer Institute (Bethesda, MD). [3H]MTX (10-30 Ci/mmol) and [³H]folic acid (32.9 Ci/mmol) were purchased from Moravek Biochemicals (Brea, CA). Cisplatin was purchased from Tocris Bioscience (Bristol, UK). The novel pyrrolo[2,3-d]pyrimidine antifolates AGF94, AGF278 and AGF283 were synthesized as previously described^{15, 16}. Additional chemicals were purchased from commercial sources in the highest available purities.

EOC cell lines and antiproliferative experiments-BR-5 and BR-Luc murine EOC cells were generous gifts from Dr. Sandra Orsulic (UCLA)^{18, 36}. KB nasopharyngeal carcinoma and SKOV3 cells were obtained from the American Type Culture Collection (Manassas, VA). IGROV1 (NCI-IGROV1) (passage 5) clear cell carcinoma cells were obtained from the Division of Cancer Treatment and Diagnosis, National Cancer Institute (Frederick, MD). A2780 and A2780-E80 were generous gifts from Dr. Gen-Sheng Wu (Karmanos Cancer Institute, Detroit, MI). All the cell lines were cultured at 37° C. under 5% CO₂ in complete folate-free RPMI 1640 supplemented with 10% fetal bovine serum (FBS) (Sigma-Aldrich; St. Louis, MO) and 100 units/mL penicillin/100 μg/mL streptomycin, and 2 mM L-glutamine. All cell lines were authenticated by STR analysis by Genetica DNA Laboratories (Burlington, NC) and tested for Mycoplasma by PCR using a Mycloplasma testing kit (Venor™ GeM Mycoplasma Detection Kit, Sigma). Frozen stocks were generated from authenticated mycoplasma-free cultures.

Cell proliferation assays were performed as described^{6, 21}. Cells were plated in 96-well dishes at densities ranging from 2500-5000 cells/well in 200 μL media and treated with a range of inhibitors spanning 0-1000 nM. Experiments used BR-5 and BR-Luc cells and folate-free RPMI 1640 media with 10% dialyzed FBS and 100 units/mL penicillin/100 μg/mL streptomycin, supplemented with 25 nM leucovorin and 2 mM L-glutamine. FR-mediated drug uptake was assessed in parallel incubations including 200 nM folic acid. Cells were treated over a 96 h period at 37° C. with 5% CO₂ and relative cell numbers were quantified using the CellTiter-blue cell viability assay (Promega, Madison, WI) and a fluorescence plate reader²¹. Raw data were exported to Excel for analysis and the results plotted using Graphpad Prism 6.0. Determinations of IC₅₀s were made corresponding to the drug concentrations that resulted in 50% loss of cell growth.

Real-time RT-PCR of transcripts for the major folate transporters and C1 metabolic targets-RNAs were isolated from the murine (BR-5, BR-Luc) and human (IGROV1, SKOV3, A2780 E-80, and A2780) EOC cell lines using TRIzol reagent (Life Technologies). cDNAs were synthesized with random hexamers and MuLV reverse transcriptase (including RNase inhibitor; Applied Biosystems) and purified using a QIAquick PCR Purification Kit (Qiagen).

Human cDNAs were purchased from Origene (HORT502) containing 48 lyophilized cDNAs from EOC patient specimens (8 stage I, 9 stage II, 17 stage III, and 6 stage IV) and 8 cDNAs from normal ovaries. Patient pathology characteristics are set forth in Table 2.

Quantitative real-time RT-PCR was performed using a Roche LightCycler 480 (Roche Diagnostics) with gene-specific primers for mouse PCFT, RFC, GARFTase, AICARFTase, and FRa, or human GARFTase and AICARFTase, as appropriate (Supplemental Table 1), and FastStart DNA Master SYBR Green I Reaction Mix (Roche Diagnostics, Indianapolis, IN). Transcript levels were normalized to β-actin transcripts. For the murine transcripts, levels were normalized to levels in mouse liver.

[³H]Folic acid binding as a measure of surface FRs—Total FRa levels were measured for the KB, IGROV1, SKOV3, BR-5, and BR-Luc cells with a functional readout involving measuring [3H]folic acid binding to surface FRs⁶. Cells were plated at a density of 1-2×10⁶ cells in complete folate-free RPMI1640 (10% FBS) media. Cells were allowed to adhere to the plates for 24 h. The following day, the cells were washed (3×) with 4° C. Dulbecco's phosphate-buffered saline (DPBS). Cells were washed with acetate buffer (10 mM sodium acetate, 150 mM NaCl, pH 3.5) (3×) at 4° C. to release FRa-bound folates, then neutralized with Hepes-buffered saline (HBS) (20 mM Hepes, 140 mM NaCl, 5 mM KCl, 2 nM MgCl₂, and 5 mM glucose, pH 7.4) at 4° C. The cells were incubated at 0° C. for 15 min with [3H]folic acid (50 nM, specific activity 0.5 Ci/mmol), in the presence or absence of non-radioactive folic acid as a competitor (10 μmol/L). After a 15 min incubation, the cells were washed with ice-cold HBS and proteins were solubilized with 0.5 N NaOH. Cell homogenates were assayed for radioactivity with a liquid scintillation counter and protein concentrations were measured using the Folin-phenol reagent³⁷. Levels of [3H]folic acid bound to FRs were expressed as pmol [³H]folic acid/mg protein.

PCFT transport assays-PCFT transport assays were performed with IGROV1, BR-5 and BR-Luc cells. Cells were plated in 60 mm dishes containing complete folate-free RPMI 1640 with 10% FBS, including 2 mmol/L L-glutamine, and antibiotics; cultures were used when they were 80-90% confluent. Uptake of [³H]MTX (at 0.5 μmol/L) was measured over 5 min at 37° C. in MES-buffered saline (20 mmol/L MES, 140 mmol/L NaCl, 5 mmol/L KCl, 2 mmol/L MgCl₂, and 5 mmol/L glucose; pH 5.5). Under these conditions, uptake by FRs is negligible. To quench transport fluxes, dishes were washed 3 times with ice-cold DPBS. Cells were solubilized in 0.5 N NaOH and radioactive contents and protein concentrations (33) determined. Uptake was expressed as pmol [3H]MTX per mg protein. To confirm PCFT-mediated transport activity, 10 μmol/L nonradioactive AGF94 was added to the transport incubations to block PCFT uptake. In vivo studies—The mouse studies were approved by the Wayne State University Institutional Animal Care and Use Committee (IACUC). Syngeneic female FVB mice were purchased from Charles River Labs (Wilmington, MA) or Envigo (Indianapolis, IN). In vivo tumor maintenance and drug efficacy studies for SC BR-Luc tumors are analogous to those previously described^{6, 15, 16, 19}.

For the in vivo therapeutic trials, study mice were maintained on either a folate-deficient diet from HarlanTeklad (Envigo, Indianapolis, IN) (catalog #TD.00434) or a folate-replete diet from Lab Diet (catalog #5021; autoclavable mouse breeder diet) (for both SC and intraperitoneal (IP) trials) starting 9-14 days before tumor implant depending on tumor staging. Mice were supplied with food and water ad libitum. Serum folate levels were determined prior to tumor implant and post study via Lactobacillus casei bioassays³⁸.

The BR-Luc tumor was first established subcutaneously from cultured cells with implanted donor mice used to set up the efficacy studies. BR-Luc tumors were aseptically harvested, mechanically dissociated into single cell suspensions, centrifuged and suspended in sterile chilled saline at a titer of 5×10⁶/0.2 ml/mouse) injected IP on day 0. The mice were subsequently unselectively distributed into control and AGF94-treated groups (4 mice/group). Treatment (IV tail vein; 0.2 ml volume) with AGF94 was initiated 4 days post-tumor implantation at 32 mg/kg injection on a Q4dx4 schedule (total dose of 128 mg/kg). Mice were weighed and observed daily for symptoms from drug treatment and disease onset (abdominal distention, palpable internal masses, concomitant with periodic monitoring of internal progression via bioluminescent imaging for tagged BR-Luc). To evaluate the qualitive efficacy of AGF94 treatment, mice were imaged 24 hours after receiving 2, 3, and 4 doses of drug. Imaging was performed with a Bruker CareStream in vivo Xtreme (Billerica, MA). Mice were injected IP with 150 mg/kg of XenoLight D-luciferin bioluminescent substrate (Perkin Elmer; catalog #122799). Imaging was initiated 10 min after D-luciferin injection after anesthetization with isoflurane with 2% oxygen (Fluriso; VetOne) (3% for induction and 1.5% for maintenance). For the IP study, mice were euthanized at defined disease endpoints (>2 ml ascites, internal cumulative tumor mass up to 1 g or at onset of lethargy or respiratory impairment).

For the SC efficacy trial, mice were implanted SC bilaterally via sterile 12-gauge trocar with 30 mg tumor fragments aseptically harvested from tumor donor mice on day 0. Tumors were measured every 3-4 days. On day 7, when tumor burdens approached a 350-450 mg volume range (by caliper; representative of advanced stage disease), the mice were non-selectively distributed into control and treatment groups with parallel cohorts included for imaging and analysis of immune infiltration (6-9 mice/group). Luminescent imaging was also performed on day 7 to establish a baseline for advanced disease. Treatment with AGF94 was initiated on day 8 on a Q4dx4 schedule at 32 mg/kg IV injection (days 8, 12, 16, 20). Mice were weighed and observed daily and their tumors measured by caliber 2-3 times weekly with weekly luminescent imaging occurring for parallel imaging cohorts. All mice (otherwise symptomatic) were euthanized at harvest or study endpoint when the tumor burden reached 5-10% body weight. Tumor volumes expressed in mg were calculated using the formula, length×width²/2 (ascertained from caliper measurements in mm). Total tumor burden per mouse (expressed in mg) was determined by addition of tumor volumes on the right and left flanks. Median tumor burden was determined on each measurement day for each group. These values were used in the efficacy analysis summarized below.

Quantitative endpoints for the IP study include tumor growth delay (T-C) using the median survival time (median time of sacrifice) in days for treated (T) and control (C) groups and the percent increase in lifespan (% ILS) using the formula T-C/C×100. Log cell kill calculations were performed as described for the SC study below. Tumor volume doubling times (Td) were determined by best fit straight line from log-linear growth plot of control group tumors in log growth phase (100-800 mg for SC tumors) or by the difference in median survival times in days of titered no treatment controls (i.e., 10⁶ and 10⁴ cells, utilizing the formula Td=median survival in days for 10⁶-median survival time for 10⁴/3.32×2).

Quantitative endpoints for the SC study include: (i) tumor growth delay [T-C, where T is the median time in days required for the treatment group tumors to reach a predetermined size (e.g., 1000 mg), and C is the median time in days for the control group tumors to reach the same size; tumor-free survivors are excluded from these calculations]; (ii) gross log₁₀ cell kill (LCK), determined by the formula LCK=(T-C; tumor growth delay in days)/3.32×Td (tumor doubling time in days determined by growth plot). Qualitative assessment of efficacy as determined by (iii) T/C values (in percent), corresponding to periodic caliper measurements utilizing the median tumor burden for treatment (T) and control (C) groups when control tumors were still in exponential growth phase (i.e., 500-1250 mg).

Tumor and spleen dissociation and flow cytometry analysis of immune populations—After 2, 3, or 4 treatments of AGF94 for the SC treatment trial, mice from the respective study arms were euthanized and tumors and spleens were dissociated. Tumors were dissociated using a GentleMACS dissociator (Miltenyi Biotec, Bergisch Gladbach, Germany) and the manufacturer's protocol for mouse tumor dissociation. Following dissociation, tumors and associated immune infiltrate cells were resuspended with 1.5% FBS in 1× DPBS for flow cytometry.

Frosted glass slides were used to dissociate spleens in 1×DPBS. Following dissociation, cells were centrifuged at 4° C. for 5 min at 1500 rpm. Red blood cells were lysed with 1 mL of H₂O, followed by the addition of 1 mL of 2×DPBS, and the supernatant was transferred to a new conical tube for centrifugation. The splenocytes were resuspended with 1.5% FBS in 1× DPBS for flow cytometry.

Flow cytometry was performed using a Becton Dickenson LSR II SORP (405/488/561/640) (BD Biosciences, San Jose, CA) and data analysis was carried out using FCS Express (De Novo Software, Pasadena, CA). For macrophage analysis, the following antibody panel was used: FRβ-eFluor660-APC (Biolegend; 153305); F4/80-APC-R700 (BD-Horizon; 565787); CD80-PE-Cy5 (Invitrogen; 15-0801-81); ghost viability dye Violet 510 (TONBO biosciences, 13-0870-T100); BV605-conjugated CD45 (clone 30-F11, BD Biosciences, 563053CD11b-PE-CF594 (BD Horizon; 562317); and Arg1-PE-Cy7 (Invitrogen; 25-3697-82).

For analysis of T cell populations, the following antibody panel was used: BV605-conjugated CD45 (clone 30-F11, BD Biosciences, 563053); APC efluor 780-conjugated CD3 (clone 17A2, Invitrogen, 47-0032-82); PerCP-Vio700-conjugated CD8a (clone 53-6.7, BD Bioscenices, 566410); Alexa Fluor-488-conjugated CD4 (clone RM4-5, Biolegend, 100532); and ghost viability dye Violet 510 (TONBO biosciences, 13-0870-T100).

Infiltrating macrophages were gated based on viable CD45+/CD11b+/F4/80 cells. M2-like FRβ positive macrophages were described as CD45+/CD11b+/F4/80+/Arg1+/FRβ. M1-like FRB positive macrophages were described as CD45+/CD11b+/F4/80+/CD80+/FRβ. Infiltrating T cells were gated based on viable CD45+/CD3+ cells. Infiltrating CD4+ and CD8+ T cells were gated based on viable CD45+/CD3+/CD4+ or CD8+ cells. The percentage of infiltrating CD3+, CD4+, CD8+, total macrophages, total FRβ macrophages, M1-like macrophages and M2-like macrophages were compared between control mice and AGF94-treated mice.

Immunofluorescence—For immunofluorescence staining, harvested tumors were formalin fixed, embedded in paraffin, and tissue slides were cut at a thickness of 4 microns. Tissue sections were processed through a series of washes with xylenes, 95% alcohol and distilled water. Sections were processed by heat-induced epitope retrieval using 1× citrate, pH 6 (Vector Lab, catalog #: H-3300-250), 1× PT Module buffer (Abcam, catalog #: ab96384), or Triology (EDTA based, pH 8.0 buffer, GeneTex) in a decloaking chamber (Biocare Medical). Tissue sections were stained for the following antibodies overnight at 4° C. in a humidity chamber: CD3 (1:100, PT buffer, Cell Signaling, catalog #: 99940); FRβ (1:500, Citrate, Triology, Genetex, catalog #: GTX105822); and CD8 (1:50, Citrate, Cell Signaling, catalog #: #98941). Slides were washed with DPBS and incubated with secondary antibodies for 1 hour with goat anti-rabbit IgG (H+L) Alexa Fluor 647 (1:200, Thermo Fisher, Catalog #A-21245). Control slides were incubated with goat anti-rabbit IgG (H+L) Alexa Fluor 647 (Supplementary Figure S1). 4′,6-Diamidino-2-phenylindole (DAPI) was used as a counterstain. Sections were mounted and imaged using a Zeiss LSM 780 confocal microscope at 63× oil magnification. Images were processed and compiled using ZEN lite (Zeiss). Western blots of FRβ-expressing Chinese hamster ovary (CHO) cells with FRβ antibody. MTXRIIOua^{R}2-4 (RFC-, PCFT- and FRa-null Chinese hamster ovary) cells (R2) were a gift from Dr. Wayne Flintoff (University of Western Ontario)⁵². Isogenic CHO cell lines were subsequently derived from R2 cells by transfection with FRα (RT16 cells) or FRβ (D4 cells) cDNAs. The CHO sublines were grown in α-minimal essential medium (α-MEM) supplemented with 100 units/mL penicillin/100 μg/mL streptomycin, 2 mM L-glutamine and 10% bovine calf serum (Sigma-Aldrich). For western blots, cells (˜ 5×10⁷) were disrupted by sonication and cell debris removed by centrifugation (1,800 rpm, 5 min). A particulate membrane fraction was prepared by centrifugation at 37,000×g. The membrane pellet was solubilized with 1% SDS in 10 mM Tris-HCl, pH 7 (containing protease inhibitors; Roche Diagnostics). Membrane proteins (28 μg) were electrophoresed on 10% Tris/glycine gels with S DS⁵³ and transferred to polyvinylidene difluoride membranes (Thermo Scientific, Rockford, IL). The membrane was probed with FRβ antibody (1:500, Genetex, Catalog: GTX105822); detection was with IRDye700CW-conjugated goat anti-rabbit IgG secondary antibody (LI-COR Biosciences, Lincoln, NE). Membranes were scanned with an Odyssey® infrared imaging system (LI-COR Biosciences, Omaha, NE). Protein loading was normalized to levels of β-actin using a β-actin mouse antibody (Sigma-Aldrich).

Statistical analyses-All data reflect at least three biological replicates unless noted otherwise. For in vitro cell based assays, expression levels were assessed using Welch's unpaired t-test after log 2 transformation to meet the normality assumption. For transcript analysis, a nonparametric Wilcoxon rank sum test was performed. For survival curves between control and AGF94 treated mice with IP BR-Luc tumors, a Log-rank (Mantel-Cox) test was performed. For flow cytometry, a nonparametric Wilcoxin rank test was performed. Statistical analyses were performed using Excel and Graphpad Prism 6.0.

### Comparative Data—not all Substituted Pyrrolo[2,3-d]Pyrimidines are FRβ Selective

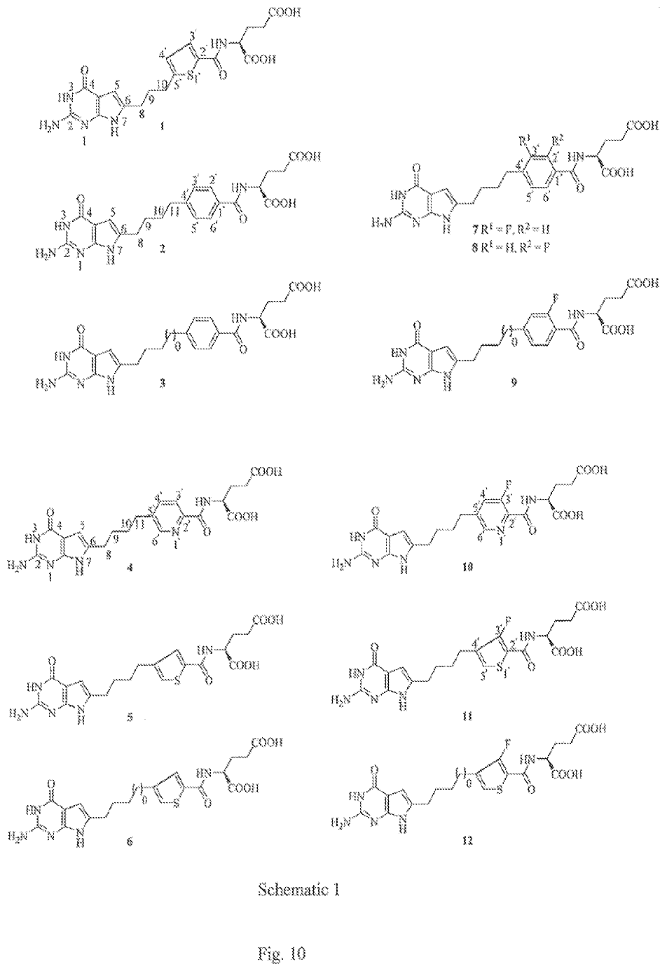

Several successful clinically useful cancer chemotherapy agents target folate and nucleotide metabolism, demonstrating the importance of these one-carbon metabolism pathways to the malignant phenotype. Pemetrexed (PMX) a substituted pyrrolo[2,3-d]pyrimidine that is a successful agent used in treating hematologic malignancies and solid tumors. Although clinical responses to therapy are notable, drawbacks of PMX include severe toxicities and drug resistance, resulting in treatment failure. The causes of drug toxicity are complex but invariably reflect a lack of selectivity for tumors over normal cells. The reduced folate carrier (RFC) is one of three principal transporters for cellular uptake of folate cofactors and classical antifolates into mammalian cells, the others being the protoncoupled folate transporter (PCFT) and folate receptors (FRs) a and B. While RFC is the major mechanism for cellular uptake of PMX and is an important determinant of clinical antitumor efficacy, RFC does not provide for tumor-selective uptake of cytotoxic folate-based analogues, as RFC is abundantly expressed in normal tissues as well as tumors. For this reason, the present applicants have found that not all substituted pyrrolo[2,3-d]pyrimidines are FRβ selective enough to inhibit TAMS in a cancer patient. Table 3 shows comparative FRβ inhibitory data for certain substituted pyrrolo[2,3-d]pyrimidines (structures set forth below in FIG. 10-Schematic 1). Table 3 shows that compounds 1, 2, 3 (AGF94) 4, 5, 6, 8, 9, 10, 11 (AGF278), and 12 (AGF283) are at least ten times better transported by folate receptor beta compared with pemetrexed (PMX). This clearly indicates that the methods using the present compounds are at least ten times better at penetrating TAMS than PMX. Thus, those persons of ordinary skill in the art will understand that not all substituted pyrrolo[2,3-d]pyrimidines (i.e, PMX) will have efficacy in the methods of this invention. FIG. 10-Schematic 1 shows the structures of the pyrrolo[2,3-d]pyrimidines that have efficacy in the methods of the present invention.

TABLE 1
IC₅₀ values (in nM) for anti-proliferative activities of novel compounds
on HGSOC mouse models BR-Luc and BR-5 compared to cisplatin.

| | BR-Luc | | BR-5 | | IGROV1 | |
|---|---|---|---|---|---|---|
| | No | Folic Acid | | Folic Acid | No | Folic Acid |
| Inhibitor | addition | (200 nM) | No addition | (200 nM) | addition | (200 nM) |
| Cisplatin | 290 ± 170 | ND | 160 ± 110 | ND | 856.67 ± 62.27 | ND |
| AGF94 | 5.86 ± 3.67 | 249 ± 123.48 | 8.10 ± 4.90 | 346 ± 165.59 | 4.14 ± 0.94 | 194.56 ± 29.3 |
| AG278 | 6.06 ± 3.12 | 588.34 ± 162.50 | 6.55 ± 3.24 | 546.94 ± 188.08 | 1.90 ± 0.15 | 426 ± 152 |
| AG283 | 75.52 ± 33.28 | 639.42 ± 164.97 | 62.89 ± 13.76 | 597.7 ± 158.12 | 5.61 ± 1.20 | 241.8 ± 106.0 |

Proliferation assays were performed for BR-5 and BR-Luc mouse HGSOC tumor cells (both express RFC, FRα, and PCFT). Anti-proliferative studies were performed in folate-free RPMI1640 media with 10% dialyzed FBS serum and 25 nM leucovorin. To measure FR-mediated drug delivery, experiments were performed in the presence or absence of 200 nM folic acid. Results are presented as IC₅₀ values, corresponding to the concentrations that inhibit growth by 50% relative to cells incubated without drug. The data are presented as mean values from at least 3 experiments (+/− standard errors in parentheses). The pyrrolo[2,3-d]pyrimidine antifolates were previously described^{15, 16}. ND, not done.

TABLE 2
Epithelial ovarian cancer (EOC) tissue cDNA array for real-time RT-PCR. The
cDNA array (Hort502) (Origene, Inc) contained 48 samples, including 8 normal,
8 stage I, 9 stage II, 17 stage III, and 6 stage IV EOC. Age, pathology,
diagnosis, tumor grade and stage for each patient are summarized below.

| Specimen | Age | Pathology | Diagnosis | Tumor grade | Stage |
|---|---|---|---|---|---|
| 1 | 46 | Normal | Leiomyoma of myometrium | Not Applicable | 0 |
| 2 | 49 | Normal | Leiomyoma of myometrium | Not Applicable | 0 |
| 3 | 46 | Normal | Leiomyoma of myometrium | Not Applicable | 0 |
| 4 | 42 | Normal | Endometrium, secretory | Not Applicable | 0 |
| 5 | 33 | Normal | No residual malignancy | Not Applicable | 0 |
| 6 | 40 | Normal | Cyst of ovary, follicular | Not Applicable | 0 |
| 7 | 45 | Normal | Endometriosis | Not Applicable | 0 |
| 8 | 35 | Normal | Endometriosis | Not Applicable | 0 |
| 9 | 70 | Tumor | Carcinoma of ovary, papillary serous | FIGO G2: Poorly | IA |
| | | | | differentiated | |
| 10 | 68 | Tumor | Adenocarcinoma of ovary, papillary | FIGO G2: Moderately | IA |
| | | | cell | differentiated | |
| 11 | 48 | Tumor | Adenocarcinoma of ovary, clear cell | Not Reported | IA |
| 12 | 58 | Tumor | Adenocarcinoma of ovary, | FIGO G1: Well | IB |
| | | | endometrioid | differentiated | |
| 13 | 55 | Tumor | Adenocarcinoma of ovary, | FIGO G3: Poorly | IB |
| | | | endometrioid | differentiated | |
| 14 | 74 | Tumor | Adenocarcinoma of ovary, | FIGO G3: Poorly | IB |
| | | | endometroid, papillary serous | differentiated | |
| 15 | 65 | Tumor | Carcinoma of ovary, papillary serous | FIGO G3: Poorly | IC |
| | | | | differentiated | |
| 16 | 58 | Tumor | Carcinoma of ovary, endometrioid | FIGO G2: Moderately | IC |
| | | | | differentiated | |
| 17 | 78 | Tumor | Adenocarcinoma of ovary, papillary | FIGO G2: Moderately | IIA |
| | | | serous | differentiated | |
| 18 | 44 | Tumor | Carcinoma of ovary, papillary serous | FIGO G2: Moderately | IIA |
| | | | | differentiated | |
| 19 | 73 | Tumor | Adenocarcinoma of ovary | FIGO G3: Poorly | IIA |
| | | | | differentiated | |
| 20 | 67 | Tumor | Adenocarcinoma of ovary, serous | FIGO G2: Moderately | IIB |
| | | | | differentiated | |
| 21 | 67 | Tumor | Adenocarcinoma of ovary, serous | FIGO G2: Moderately | IIB |
| | | | | differentiated | |
| 22 | 58 | Tumor | Adenocarcinoma of ovary, | FIGO G3: Poorly | IIB |
| | | | endometrioid | differentiated | |
| 23 | 58 | Tumor | Adenocarcinoma of ovary, papillary | FIGO G2: Moderately | IIB |
| | | | serous | differentiated | |
| 24 | 59 | Tumor | Adenocarcinoma of ovary, papillary | FIGO G3: Poorly | IIC |
| | | | serous | differentiated | |
| 25 | 63 | Tumor | Adenocarcinoma of ovary, serous | FIGO G2: Moderately | IIC |
| | | | | differentiated | |
| 26 | 51 | Tumor | Adenocarcinoma of ovary, serous | FIGO G2: Moderately | III |
| | | | | differentiated | |
| 27 | 73 | Tumor | Adenocarcinoma of ovary, serous | FIGO G3: Poorly | III |
| | | | | differentiated | |
| 28 | 91 | Tumor | Adenocarcinoma of ovary, papillary | FIGO G3: Poorly | III |
| | | | serous | differentiated | |
| 29 | 75 | Tumor | Adenocarcinoma of ovary, | FIGO G3: Poorly | IIIA |
| | | | endometrioid | differentiated | |
| 30 | 65 | Tumor | Adenocarcinoma of ovary, serous | FIGO G3: Poorly | IIIA |
| | | | | differentiated | |
| 31 | 55 | Tumor | Adenocarcinoma of ovary, mucinous | FIGO G2: Moderately | IIIA |
| | | | | differentiated | |
| 32 | 66 | Tumor | Adenocarcinoma of ovary, serous | FIGO G2: Moderately | IIIA |
| | | | | differentiated | |
| 33 | 80 | Tumor | Adenocarcinoma of ovary, serous | Not Reported | IIIB |
| 34 | 64 | Tumor | Adenocarcinoma of ovary, papillary | FIGO G3: Poorly | IIIB |
| | | | serous | differentiated | |
| 35 | 66 | Tumor | Carcinoma of ovary, papillary serous | FIGO G3: Poorly | IIIB |
| | | | | differentiated | |
| 36 | 52 | Tumor | Adenocarcinoma of ovary, papillary | FIGO G3: Poorly | IIIB |
| | | | serous | differentiated | |
| 37 | 71 | Tumor | Adenocarcinoma of ovary, papillary | FIGO G3: Poorly | IIIC |
| | | | serous | differentiated | |
| 38 | 48 | Tumor | Adenocarcinoma of ovary, papillary | FIGO G2: Moderately | IIIC |
| | | | serous | differentiated | |
| 39 | 50 | Tumor | Carcinoma of ovary, papillary serous | FIGO G3: Poorly | IIIC |
| | | | | differentiated | |
| 40 | 58 | Tumor | Adenocarcinoma of ovary, papillary | FIGO G2: Moderately | IIIC |
| | | | serous | differentiated | |
| 41 | 53 | Tumor | Adenocarcinoma of ovary, serous | FIGO G3: Poorly | IIIC |
| | | | | differentiated | |
| 42 | 66 | Tumor | Adenocarcinoma of ovary, | FIGO G3: Poorly | IIIC |
| | | | endometrioid, papillary serous | differentiated | |
| 43 | 45 | Tumor | Adenocarcinoma of ovary, serous, | Not Reported | IV |
| | | | metastatic | | |
| 44 | 80 | Tumor | Adenocarcinoma of ovary, | FIGO G3: Poorly | IV |
| | | | endometroid, papillary serous | differentiated | |
| 45 | 68 | Tumor | Adenocarcinoma of ovary, serous | FIGO G3: Poorly | IV |
| | | | | differentiated | |
| 46 | 61 | Tumor | Adenocarcinoma of ovary, serous | FIGO G3: Poorly | IV |
| | | | | differentiated | |
| 47 | 60 | Tumor | Adenocarcinoma of ovary, papillary | Not Reported | IV |
| | | | serous, metastatic | | |
| 48 | 63 | Tumor | Adenocarcinoma of ovary, papillary | FIGO G3: Poorly | IV |
| | | | serous | differentiated | |

TABLE 3

| Antifolate | CHO cells |
|---|---|
| Compound | (IC₅₀ nm) |
| No. | FRβ |
| 1 | 0.16(0.02) |
| 2 | 6.3(1.6) |
| 8 | 1.6(0.44) |
| 3(AGF94) | 5.6(1.2) |
| 9 | 3.87(0.14) |
| 4 | 0.51(0.09) |
| 10 | 0.44(0.13) |
| 5 | 0.43(0.14) |
| 11(AGF278) | 0.75(0.42) |
| 6 | 0.34(0.03) |
| 12(AGF283) | 0.19(0.02) |
| PMX | 60(8) |

Those skilled in the art shall understand that chemical structures 1-12 (FIG. 10-Schematic 1) are preferred examples of the compounds of this invention and that tautomers of the structures of compounds 1-12 are also embodiments of the methods using these compounds. Those skilled in the art understand that chemical structures are often drawn as one tautomeric form over another. This invention provides for several tautomeric forms of the oxygen attached at the fourth carbon of the pyrimidine six membered ring of the compounds of this invention. The tautomeric forms (i.e. oxygen with double bond, or —OH) provide additional structural embodiments that will be appreciated by those skilled in the art.

In certain embodiments of the invention, the novel compounds, as described herein, include pharmaceutically acceptable salts of these compounds, and include for example but not limited to, hydrochloride chloride (HCl) salts (or other acids) of these compounds.

It is especially advantageous to formulate parenteral compositions in dosage unit form for case of administration and uniformity of dosage. Dosage unit form as used herein refers to physically discrete units suited as unitary dosages for the patients being treated, each unit containing a predetermined quantity or effective amount of a compound of the present invention to produce the desired effect in association with a pharmaceutical carrier. The specification for the dosage unit forms of the invention is dictated by and directly dependent on the particular compound and the particular effect, or therapeutic response, that is desired to be achieved.

Compounds of this invention, as described herein, or pharmaceutically acceptable salts, or hydrates thereof, can be administered to a patient (an animal or human) via various routes including parenterally, orally or intraperitoneally. Parenteral administration includes the following routes that are outside the alimentary canal (digestive tract): intravenous; intramuscular; interstitial, intraarterial; subcutaneous; intraocular; intracranial; intraventricular; intrasynovial; transepithelial, including transdermal, pulmonary via inhalation, ophthalmic, sublingual and buccal; topical, including dermal, ocular, rectal, or nasal inhalation via insufflation or nebulization. Specific modes of administration shall depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered to a patient shall depend on the characteristics of the patient being treated, including for example, but not limited to, the patient's age, weight, health, and types and frequency of concurrent treatment, if any, of any other chemotherapeutic agent(s), all of which is determined by the clinician as one skilled in the art.

Compounds of this invention, as described herein, or a pharmaceutically acceptable salt, or hydrate thereof, that are orally administered can be enclosed in hard or soft shell gelatin capsules, or compressed into tablets. Compounds also can be incorporated with an excipient and used in the form of ingestible tablets, buccal tablets, troches, capsules, sachets, lozenges, elixirs, suspensions, syrups, wafers and the like. These compounds can be in the form of a powder or granule, a solution or suspension in an aqueous liquid or non-aqueous liquid, or in an oil-in-water emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain the active compound, and sucrose as a sweetening agent, methyl and propylparabens as preservatives, a dye and flavoring, for example. Any material used in preparing any dosage unit form should be pharmaceutically pure and substantially non-toxic. Additionally, the compounds of this invention, as described herein, or a pharmaceutically acceptable salt, or hydrate of these compounds, can be incorporated into sustained-release preparations and formulations.

These compounds, or a pharmaceutically acceptable salt, or hydrate thereof, can be administered to the central nervous system, parenterally or intraperitoneally. Solutions of the compound as a free base or a pharmaceutically acceptable salt can be prepared in water mixed with a suitable surfactant, such as for example, but not limited to, hydroxypropylcellulose. Dispersions also can be prepared in glycerol, liquid polyethylene glycols and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include, without limitation, sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi.

Compounds of the present invention may be contained within, mixed with, or associated with, a suitable (acceptable) pharmaceutical carrier (i.e. a pharmaceutically acceptable carrier) for administration to a patient according to the particular route of administration desired. Suitable or acceptable pharmaceutical carriers refer to any pharmaceutical carrier that will solubilize the compounds of the present invention and that will not give rise to incompatibility problems, and includes any and all solvents, dispersion media, coatings, antibacterial and antifungal agents, isotonic agents, absorption delaying agents, and the like. The use of such suitable or acceptable pharmaceutical carriers is well known by those skilled in the art. Preferred carriers include sterile water, physiologic saline, and five percent dextrose in water. Examples of other suitable or acceptable pharmaceutical carriers include, but are not limited to, ethanol, polyol (such as propylene glycol and liquid polyethylene glycol), suitable mixtures thereof, or vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating a compound of this invention, in the required amount in the appropriate solvent with various of the other ingredients enumerated above, as required, followed by filtered sterilization. Generally, dispersions are prepared by incorporating the sterilized compound into a sterile vehicle that contains the basic dispersion medium and any of the other ingredients from those enumerated above. In the case of sterile powders for the preparation of sterile injectable solutions, the preferred methods of preparation are vacuum drying and freeze drying.

Pharmaceutical compositions which are suitable for administration to the nose and buccal cavity include, without limitation, self-propelling and spray formulations, such as aerosol, atomizers and nebulizers.

The therapeutic compounds, as described herein, can be administered to a patient alone or in combination with pharmaceutically acceptable carriers or as pharmaceutically acceptable salts, or hydrates thereof, the proportion of which is determined by the solubility and chemical nature of the compound, chosen route of administration to the patient and standard pharmaceutical practice.

### REFERENCES

1 Torre, L. A. et al. Ovarian cancer statistics, 2018. CA Cancer J Clin, doi: 10.3322/caac.21456 (2018).
2 Lheureux, S., Gourley, C., Vergote, I. & Oza, A. M. Epithelial ovarian cancer. Lancet 393, 1240-1253 (2019).
3 Jackman, A. L., Jansen, G. & Ng, M. in Targeted Drug Strategies for Cancer and Inflammation (eds Ann L. Jackman & Christopher P. Leamon) 93-117 (Springer US, 2011).
4 Moore, K. N. et al. Phase 1 dose-escalation study of mirvetuximab soravtansine (IMGN853), a folate receptor alpha-targeting antibody-drug conjugate, in patients with solid tumors. Cancer 123, 3080-3087 (2017).
5 Reddy, J. A. et al. Preclinical evaluation of EC145, a folate-vinca alkaloid conjugate. Cancer Res 67, 4434-4442 (2007).
6 Hou, Z. et al. Dual Targeting of Epithelial Ovarian Cancer Via Folate Receptor alpha and the Proton-Coupled Folate Transporter with 6-Substituted Pyrrolo[2,3-d]pyrimidine Antifolates. Mol Cancer Ther 16, 819-830 (2017).
7 Matherly, L. H., Hou, Z. & Gangjee, A. The promise and challenges of exploiting the proton-coupled folate transporter for selective therapeutic targeting of cancer. Cancer Chemother Pharmacol 81, 1-15 (2018).
8 Worzfeld, T. et al. The Unique Molecular and Cellular Microenvironment of Ovarian Cancer. Front Oncol 7, 24, doi: 10.3389/fonc.2017.00024 (2017).
9 Takaishi, K. et al. Involvement of M2-polarized macrophages in the ascites from advanced epithelial ovarian carcinoma in tumor progression via Stat3 activation. Cancer Sci 101, 2128-2136 (2010).
10 Colvin, E. K. Tumor-associated macrophages contribute to tumor progression in ovarian cancer. Front Oncol 4, 137 (2014).
11 Bejarano, L., Jordao, M. J. C. & Joyce, J. A. Therapeutic Targeting of the Tumor Microenvironment. Cancer Discov 11, 933-959, doi: 10.1158/2159-8290.CD-20-1808 (2021).
12 Kowal, J., Kornete, M. & Joyce, J. A. Re-education of macrophages as a therapeutic strategy in cancer. Immunotherapy 11, 677-689 (2019).
13 Puig-Kroger, A. et al. Folate receptor beta is expressed by tumor-associated macrophages and constitutes a marker for M2 anti-inflammatory/regulatory macrophages. Cancer Res 69, 9395-9403 (2009).
14 Cresswell, G. M. et al. Folate Receptor Beta Designates Immunosuppressive Tumor-Associated Myeloid Cells That Can Be Reprogrammed with Folate-Targeted Drugs. Cancer Res 81, 671-684 (2021).
15 Ravindra, M. et al. Fluorine-Substituted Pyrrolo[2,3-d]Pyrimidine Analogues with Tumor Targeting via Cellular Uptake by Folate Receptor alpha and the Proton-Coupled Folate Transporter and Inhibition of de Novo Purine Nucleotide Biosynthesis. J Med Chem 61, 4228-4248 (2018).
16 Wang, L. et al. Synthesis, biological, and antitumor activity of a highly potent 6-substituted pyrrolo[2,3-d]pyrimidine thienoyl antifolate inhibitor with proton-coupled folate transporter and folate receptor selectivity over the reduced folate carrier that inhibits beta-glycinamide ribonucleotide formyltransferase. J Med Chem 54, 7150-7164, doi: 10.1021/jm200739e (2011).
17 Sale, S. & Orsulic, S. Models of ovarian cancer metastasis: Murine models. Drug Discov Today Dis Models 3, 149-154 (2006).
18 Xing, D. & Orsulic, S. A mouse model for the molecular characterization of brca1-associated ovarian carcinoma. Cancer Res 66, 8949-8953 (2006).
19 Cherian, C. et al. Therapeutic targeting malignant mesothelioma with a novel 6-substituted pyrrolo[2,3-d]pyrimidine thienoyl antifolate via its selective uptake by the proton-coupled folate transporter. Cancer Chemother Pharmacol 71, 999-1011 (2013).
20 Wilson, M. R. et al. Targeting Nonsquamous Nonsmall Cell Lung Cancer via the Proton-Coupled Folate Transporter with 6-Substituted Pyrrolo[2,3-d]Pyrimidine Thienoyl Antifolates. Mol Pharmacol 89, 425-434 (2016).
21 Deng, Y. et al. Synthesis and discovery of high affinity folate receptor-specific glycinamide ribonucleotide formyltransferase inhibitors with antitumor activity. J Med Chem 51, 5052-5063 (2008).
22 Matherly, L. H., Wilson, M. R. & Hou, Z. The Major Facilitative Folate Transporters Solute Carrier 19A1 and Solute Carrier 46A1: Biology and Role in Antifolate Chemotherapy of Cancer. Drug Metabolism and Disposition 42, 632-649 (2014).
23 Zhao, R. & Goldman, I. D. The molecular identity and characterization of a Proton-coupled Folate Transporter—PCFT; biological ramifications and impact on the activity of pemetrexed. Cancer Metastasis Rev 26, 129-139, doi: 10.1007/s10555-007-9047-1 [doi] (2007).
24 Desmoulin, S. K., Hou, Z., Gangjee, A. & Matherly, L. H. The human proton-coupled folate transporter: Biology and therapeutic applications to cancer. Cancer Biol Ther 13, 1355-1373 (2012).
25 Wallace-Povirk, A. et al. Discovery of 6-substituted thieno[2,3-d]pyrimidine analogs as dual inhibitors of glycinamide ribonucleotide formyltransferase and 5-aminoimidazole-4-carboxamide ribonucleotide formyltransferase in de novo purine nucleotide biosynthesis in folate receptor expressing human tumors. Bioorganic & Medicinal Chemistry, 116093 (2021).
26 Tie, Y. et al. Targeting folate receptor beta positive tumor-associated macrophages in lung cancer with a folate-modified liposomal complex. Signal Transduct Target Ther 5, 6, doi: 10.1038/s41392-020-0115-0 (2020).
27 Deis, S. M. et al. Structural and Enzymatic Analysis of Tumor-Targeted Antifolates That Inhibit Glycinamide Ribonucleotide Formyltransferase. Biochemistry 55, 4574-4582 (2016).
28 Tong, X., Zhao, F. & Thompson, C. B. The molecular determinants of de novo nucleotide biosynthesis in cancer cells. Current opinion in genetics & development 19, 32-37 (2009).
29 Bronder, J. L. & Moran, R. G. Antifolates targeting purine synthesis allow entry of tumor cells into S phase regardless of p53 function. Cancer Res 62, 5236-5241 (2002).
30 Bronder, J. L. & Moran, R. G. A defect in the p53 response pathway induced by de novo purine synthesis inhibition. J Biol Chem 278, 48861-48871 (2003).
31 Hori, H. et al. Methylthioadenosine phosphorylase cDNA transfection alters sensitivity to depletion of purine and methionine in A549 lung cancer cells. Cancer Res 56, 5653-5658 (1996).
32 Bertino, J. R., Waud, W. R., Parker, W. B. & Lubin, M. Targeting tumors that lack methylthioadenosine phosphorylase (MTAP) activity: current strategies. Cancer Biol Ther 11, 627-632 (2011).
33 Hoxhaj, G. et al. The mTORC1 Signaling Network Senses Changes in Cellular Purine Nucleotide Levels. Cell Rep 21, 1331-1346 (2017).
34 Rothbart, S. B., Racanelli, A. C. & Moran, R. G. Pemetrexed indirectly activates the metabolic kinase AMPK in human carcinomas. Cancer Res 70, 10299-10309 (2010).
35 Yu, W. et al. One-Carbon Metabolism Supports S-Adenosylmethionine and Histone Methylation to Drive Inflammatory Macrophages. Mol Cell 75, 1147-1160 e1145 (2019).
36 Xing, D. & Orsulic, S. A genetically defined mouse ovarian carcinoma model for the molecular characterization of pathway-targeted therapy and tumor resistance. Proc Natl Acad Sci USA 102, 6936-6941 (2005).
37 Lowry, O. H., Rosebrough, N. J., Farr, A. L. & Randall, R. J. Protein measurement with the Folin phenol reagent. J Biol Chem 193, 265-275 (1951).
38 Varela-Moreiras, G. & Selhub, J. Long-term folate deficiency alters folate content and distribution differentially in rat tissues. J Nutr 122, 986-991 (1992).

It will be appreciated by those persons skilled in the art that changes could be made to embodiments of the present invention described herein without departing from the broad inventive concept thereof. It is understood, therefore, that this invention is not limited by any particular embodiments disclosed but is intended to cover the modifications that are within the spirit and scope of the invention, as defined by the appended claims.

What is claimed is:

1. A method of inhibiting M2-like macrophages in a patient comprising administering to a patient having ovarian cancer a therapeutically effective amount of a substituted pyrrolo[2,3-d]pyrimidine antifolate inhibitor having selective efficacy to FRβ expressing tumor cells.

2. The method of claim 1 wherein said substituted pyrrolo[2,3-d]pyrimidine antifolate inhibitor is a compound selected from the group consisting of the following structures 1-12:

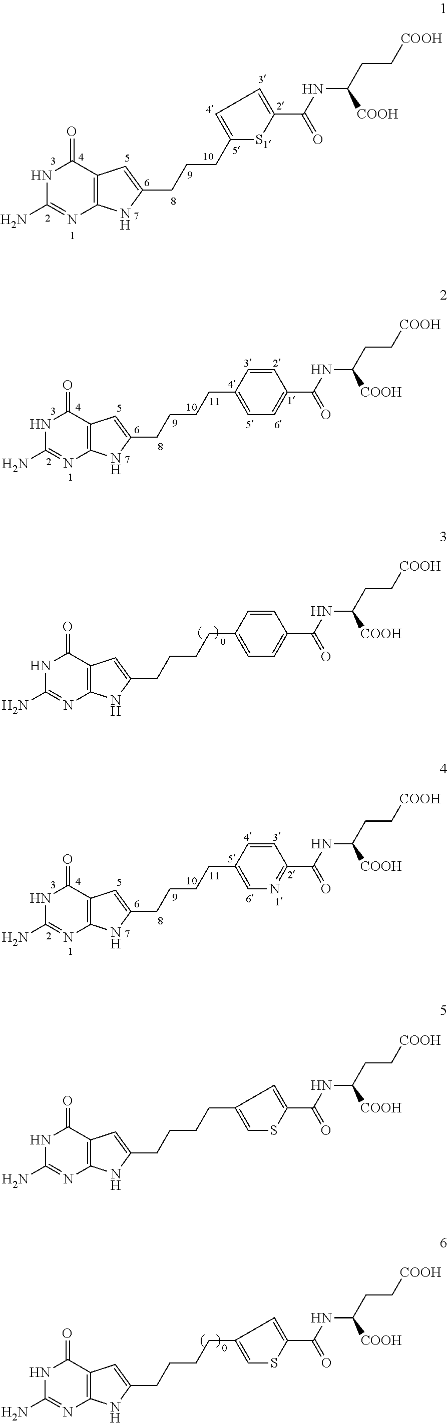

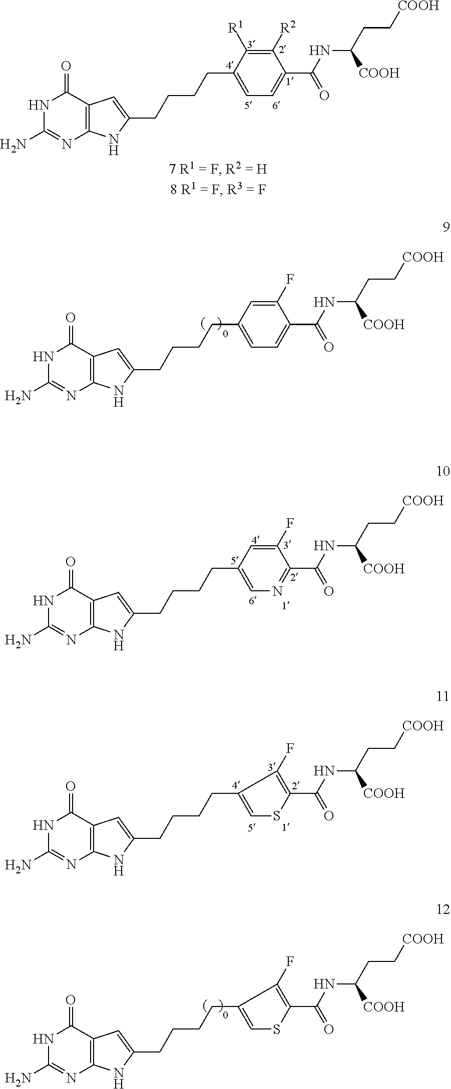

3. The method of claim 2 wherein the compound has said structure 3, 11, or 12.

4. The method of claim 1 wherein said M2-like macrophage is an M2-like FRβ-expressing macrophage.

5. The method of claim 4 wherein said M2-like FRβ-expressing macrophage is a tumor-associated macrophage (TAM).

6. The method of claim 1 wherein said administration of said compound results in anti-tumor efficacy accompanied by decreased M2-like FRβ-expressing macrophages and increased CD3+ T cells, and wherein CD4+ and CD8+ T cells are unaffected.

7. The method of claim 1 including effecting anti-tumor efficacy of said compound in the therapy of epithelial ovarian cancer (EOC) in an intact immune system and targeting an immunosuppressive tumor microenvironment (TME).

8. A method of inhibiting M2-like macrophages in a patient comprising administering to a patient having ovarian cancer a therapeutically effective amount of a composition comprising a compound that is a substituted pyrrolo[2,3-d]pyrimidine antifolate inhibitor having selective efficacy to FRβ expressing tumor cells, and an acceptable pharmaceutical carrier.

9. The method of claim 8 wherein said substituted pyrrolo[2,3-d]pyrimidine antifolate inhibitor is a compound selected from the group consisting of the following structures 1-12:

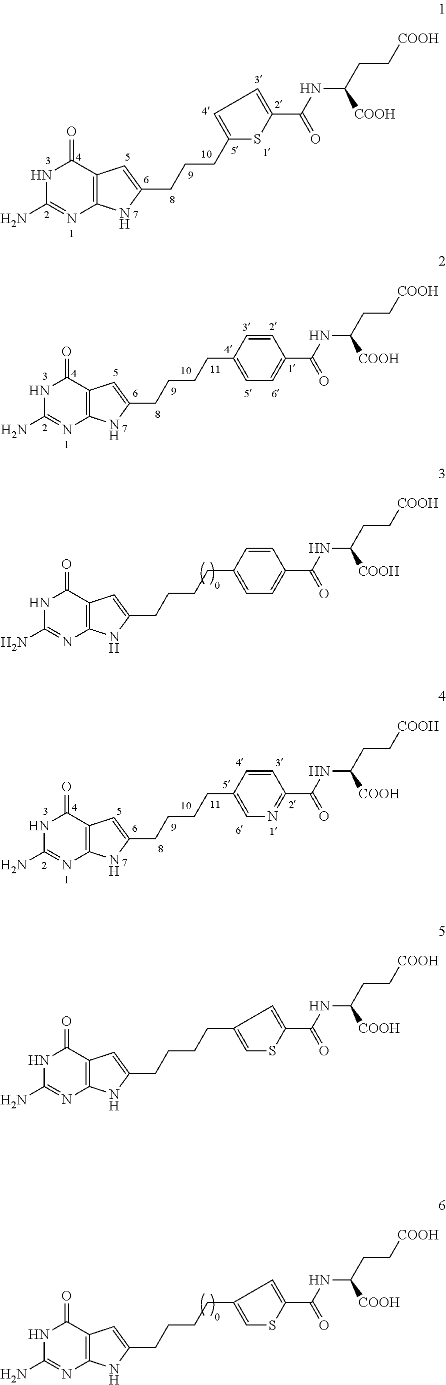

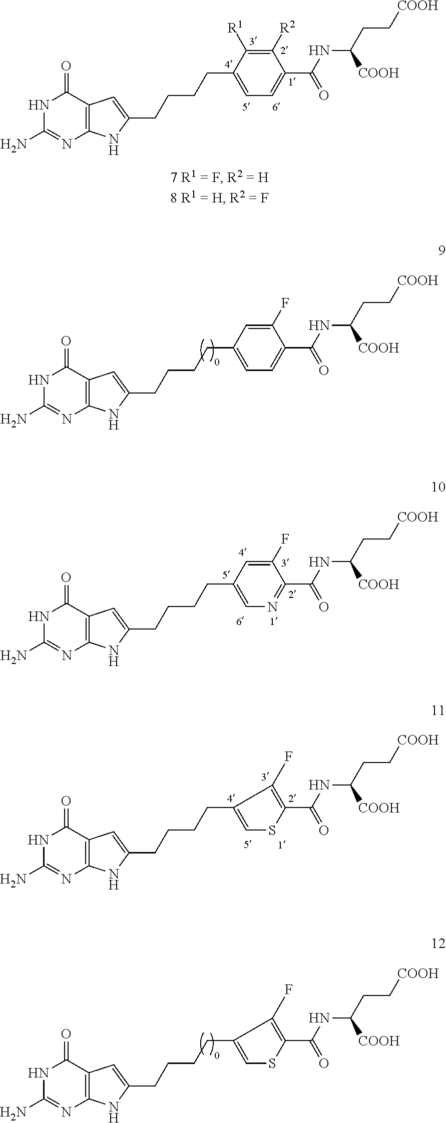

10. The method of claim 8 wherein said acceptable pharmaceutical carrier is one selected from the group of saline, dextrose and water, and sucrose.

11. The method of claim 8 including wherein said composition includes one or more pharmaceutically acceptable excipients, fillers, binders, and surfactants.

12. The method of claim 8 including effecting anti-tumor efficacy of said composition in the therapy of epithelial ovarian cancer (EOC) in an intact immune system and targeting an immunosuppressive tumor microenvironment (TME).

13. A method of inhibiting M2-like macrophages in a patient comprising administering to a patient having ovarian cancer a therapeutically effective amount of a FRβ-transported C1 inhibitor compound having selective efficacy to FRβ expressing tumor cells.

14. The method of claim 13 wherein said FRβ-transported C1 inhibitor compound is a substituted pyrrolo[2,3-d]pyrimidine antifolate inhibitor compound having selective efficacy to FRβ expressing tumor cells.

15. The method of claim 14 wherein said substituted pyrrolo[2,3-d]pyrimidine compound is a compound selected from the group consisting of the following structures 1-12:

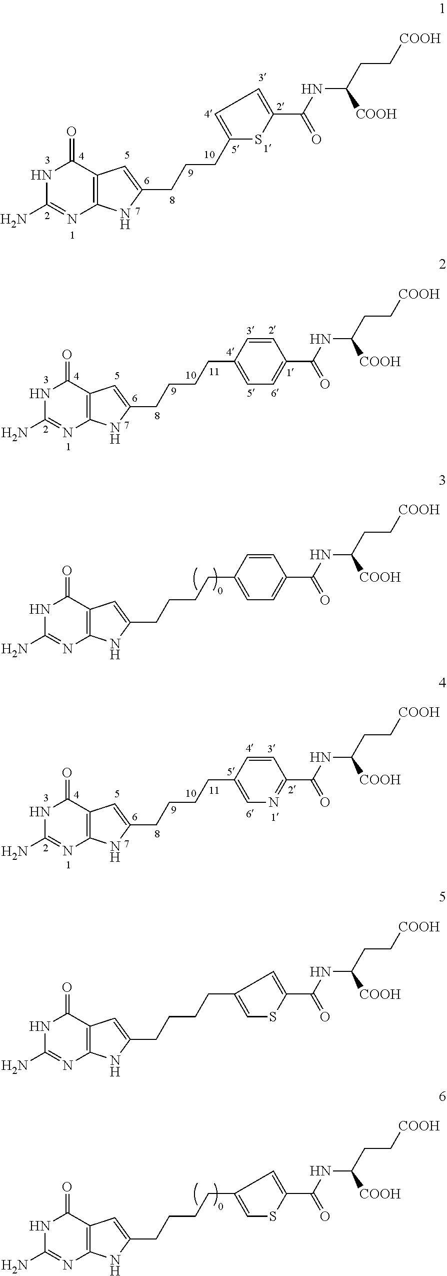

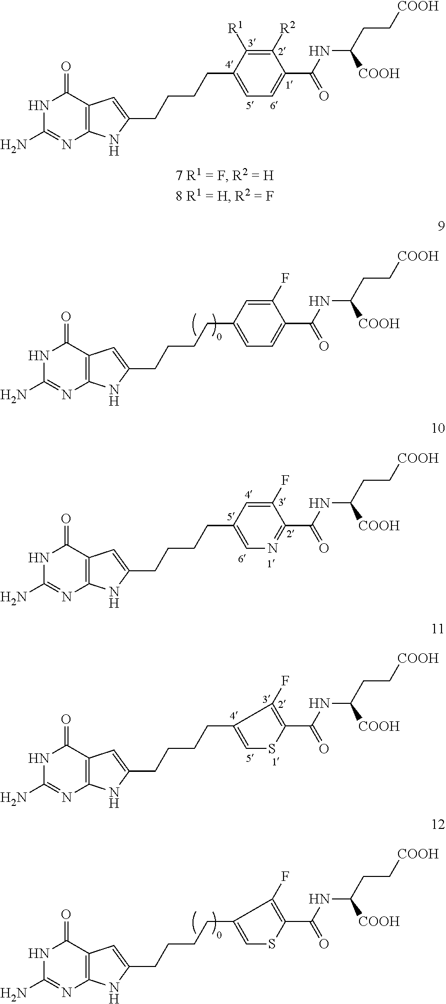

16. The method of claim 13 including effecting anti-tumor efficacy of said compound in the therapy of epithelial ovarian cancer (EOC) in an intact immune system and targeting an immunosuppressive tumor microenvironment (TME).

17. A method of inhibiting M2-like macrophages in a patient comprising administering to a patient having ovarian cancer a therapeutically effective amount of a composition comprising a FRβ-transported C1 inhibitor compound having selective efficacy to FRβ expressing tumor cells and a pharmaceutically acceptable carrier.

18. The method of claim 17 wherein said pharmaceutically acceptable carrier is one selected from the group of saline, dextrose and water, and sucrose.

19. The method of claim 17 including wherein said composition includes one or more pharmaceutically acceptable excipients, fillers, binders, and surfactants.

20. A FRβ-transported C1 inhibitor compound having selective efficacy to FRβ expressing tumor cells for use in inhibiting M2-like macrophages for treating a patient having ovarian cancer.

21. The FRβ-transported C1 inhibitor compound having selective efficacy to FRβ expressing tumor cells of claim 20 that is selected from the group consisting of substituted pyrrolo[2,3-d]pyrimidine antifolate inhibitor compounds having selective efficacy to FRβ expressing tumor cells.

22. The FRβ-transported C1 inhibitor compound having selective efficacy to FRβ expressing tumor cells of claim 21 that is a substituted pyrrolo[2,3-d]pyrimidine antifolate inhibitor compound selected from the group consisting of the following structures 1-12: 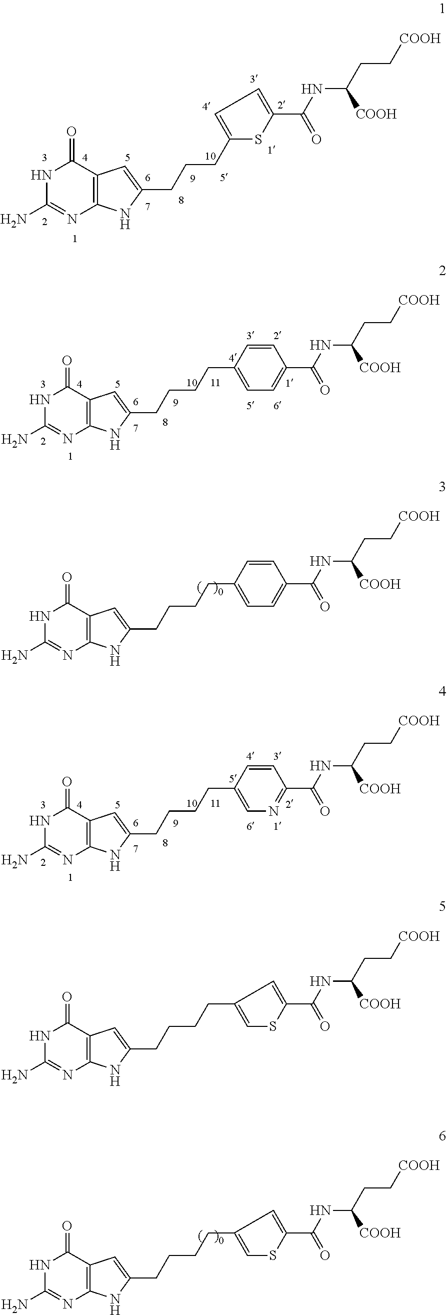 [chemical structure]
